(19) United States
(12) Patent Application Publication
(10) Pub. No.: US 2024/0417374 A1
(43) Pub. Date: Dec. 19, 2024

(54) SMALL MOLECULE ANTAGONIST TO PACAP RECEPTOR AND USES THEREOF

(71) Applicant: The University of Vermont and State Agricultural College, Burlington, VT (US)

(72) Inventors: Victor May, Essex, VT (US); Matthias Brewer, Williston, VT (US); Jianing Li, West Lafayette, IN (US)

(73) Assignee: The University of Vermont and State Agricultural College, Burlington, VT (US)

(21) Appl. No.: 18/699,084

(22) PCT Filed: Oct. 7, 2022
(86) PCT No.: PCT/US2022/046113

§ 371 Date: Apr. 5, 2024

Related U.S. Application Data
(60) Provisional application No. 63/253,862, filed on Oct. 8, 2021.

(51) Int. Cl. C07D 209/42; A61K 31/63; C07D 235/24

(52) U.S. Cl. CPC C07D 209/42; A61K 31/63; C07D 235/24

(57) ABSTRACT

The disclosure relates to compounds that demonstrate efficacy in blocking PACAP receptors (e.g. PAC1) and associated methods of treating neurological diseases or disorders using the disclosed compounds. The neurological diseases or disorders include, but are not limited to, stress-related diseases (e.g. chronic stress, post-traumatic stress disorder (PTSD), panic disorder, anxiety, or general anxiety disorder), pain-related diseases (e.g. chronic pain, primary headache disorders, or migraines), or addiction (e.g. addiction to a substance (e.g. addiction to cocaine, amphetamines, methamphetamine, methylphenidate, nicotine, alcohol, prescription medication, marijuana, tobacco, or an opioid selected from heroin, fentanyl, codeine, hydrocodone, morphine, oxycodone, hydromorphone, and methadone)).

### RELATED APPLICATIONS

This application claims priority under 35 U.S.C. § 119(e) to U.S. Provisional Application, U.S. Ser. No. 63/253,862, filed on Oct. 8, 2021, which is incorporated herein by reference.

### STATEMENT OF GOVERNMENT SUPPORT

This invention was made with government support under RO1-GM129431 awarded by National Institutes of Health. The government has certain rights in the invention.

### BACKGROUND OF THE INVENTION

Pituitary adenylate cyclase activating polypeptide (PACAP) is a widely expressed peptide in central and peripheral neurons, and behaves as a neurotransmitter and neurotrophic regulator with roles in signaling, physiological homeostasis, development, survival, proliferation, differentiation, and regeneration (Vaudry et al., 2009). PACAP belongs to the vasoactive intestinal peptide (VIP)/secretin/glucagon family of related peptides. It is endoproteolytically cleaved from a 176 amino acid precursor molecule into two alternatively processed α-amidated bioactive PACAP38 or PACAP27 peptides, but PACAP38 is typically more than 100-fold more abundant than PACAP27 in nervous tissues (Miyata et al., 1989, 1990; Arimura et al., 1991). PACAP is well conserved in evolution; PACAP can be found in tunicates and the PACAP amino acid sequence is identical in all mammals examined to date (Sherwood et al., 2000).

Because PACAP shares nearly 70% amino acid homology with VIP, the PACAP/VIP peptides share three receptor subtypes (Harmar et al., 2012; Blechman and Levkowitz, 2013). The PACAP/VIP receptors belong to Class B of the 7-transmembrane G protein-coupled receptors. PACAP binding is specific at the PAC1 receptor (ADCYAP1R1); PACAP and VIP bind with near equal high affinities to VPAC1 (VIPR1) and VPAC2 (VIPR2) receptors. There are several alternatively spliced forms of the PAC1 receptor based on the absence or presence of two 84-base pair cassettes, termed hip and hop, inserted into the receptor DNA corresponding to the third intracellular cytoplasmic loop (Spengler et al., 1993). Hence the PAC1 receptor isoforms may be null (neither hip nor hop cassette inserts), hip, hop (there is a shortened form of hop called hop2), or hip+hop. Depending on the cellular expression of the PAC1 receptor isoform, the receptor may be differentially coupled to adenylyl cyclase, phospholipase C, MAPK or Akt pathways, among others. There are also PAC1 receptor splice variants in the N-terminal extracellular domain of the receptor which may impact receptor function and activation. Splice variants have not been described for the VPAC1/VPAC2 receptors. The VPAC1 and VPAC2 receptors appear to be preferentially coupled to adenylyl cyclase. Recent work has shown that β-arrestin-mediated PAC1 receptor internalization and endosomal signaling is the primary driver of long term cellular ERK activation.

Over the last several years, the PACAP/PAC1 receptor system has been associated with stress-related anxiety-like behavior (Hammack et al., 2009; Stroth and Eiden, 2010; Roman et al., 2014; Missig et al., 2014, 2017; Lezak et al., 2014; Miles et al., 2018), including post-traumatic stress disorder (PTSD, Ressler et al., 2011; Almli et al., 2013; Jovanovic et al., 2013; Uddin et al., 2013; Wang et al., 2013; Chen et al., 2013; Pohlack et al., 2015; Hammack and May, 2015). In addition, chronic stress increases PACAP and PAC1 receptor transcripts in the bed nucleus of the stria terminalis (BNST) and hypothalamus (Hammack et al., 2009), two limbic brain regions that participate in stress responses and behavior. Infusions of PACAP or maxadilan (a specific PAC1 receptor agonist) into the BNST are anxiogenic and anorexic (analogous to stress-mediated decreases in feeding behavior) (Roman et al., 2014). Similar anxiogenic responses were elicited following PACAP infusions directly into the central amygdala (CeA, Missig et al., 2014; 2017). Conversely, antagonism of PAC1 receptor signaling with PACAP6-38 (N-terminally truncated form of PACAP—see below) during chronic stress blocked both anxiety-related and diminished feeding behaviors (Roman et al., 2014). Further, elevated blood PACAP levels and polymorphism in the estrogen responsive element (ERE) of the PAC1 receptor gene have been associated with PTSD symptoms in women (Ressler et al., 2011). These observations have been reproduced independently in other laboratories (Hammack and May, 2015; Almli et al., 2013; Jovanovic et al., 2013; Uddin et al., 2013; Wang et al., 2013; Chen et al., 2013; Pohlack et al., 2015).

PACAP is expressed in a population of sensory dorsal root ganglion neurons and can be induced upon injury or inflammation paradigms. More recently, our laboratories have shown that PACAP in nociceptive (pain) spino-parabrachioamygdaloid pathway is important in mediating the emotional components of pain (Missig et al., 2014; 2017). Briefly, PACAP in the peripheral pain circuits relay signals that ultimately terminate in the lateral capsular division of the amygdala (CeLC), a limbic nucleus well studied for its roles in stress and fear (Rouwette et al., 2012; Gauriau and Bernard, 2002). Stress and chronic pain (including migraine) are comorbid. Nearly 50% of patients with psychopathologies have chronic pain; conversely, 30-40% of patients with chronic pain present anxiety disorders including PTSD (Otis et al., 2003; McFarlane et al., 1994; McWilliams et al., 2003; Asmundson and Katz, 2009; Moeller-Bertram et al., 2012; Norman et al., 2008; Scioli-Salter et al., 2015). Hence this particular PACAP neurocircuit may represent a means by which chronic pain can induce maladaptive neuroplasticity in limbic neurons leading to behavioral abnormalities including anxiety disorders, panic, generalized anxiety disorder, and PTSD. PACAP infusions into the amygdala increase anxiety-like responses and nociceptive sensitivity; these responses can be blocked in in vivo models of chronic pain using a PACAP6-38 PAC1 receptor antagonist (Missig et al., 2017).

The behavioral and chronic pain responses are dependent on cellular ERK activation. Stress and chronic pain paradigms increase phosphorylated ERK signaling in the brain; conversely, stress and chronic pain responses can be attenuated by inhibitors of the MEK/ERK signaling cascade (Carrasquillo and Gereau, 2007). PACAP infusions into limbic areas increase ERK activation and c-fos induction (a marker for increased neuronal activity) in parallel with behavioral and pain responses; these PACAP responses can be attenuated with MEK/ERK inhibitors (Missig et al., 2017). PAC1 receptor-mediated neuronal excitability has been shown to be regulated by ERK mechanisms, notably following PAC1 receptor internalization and endosomal signaling (May et al., 2014; May and Parsons, 2017; Johnson et al., 2020a, 2020b, 2021). In coherence, inhibitors of endocytic mechanisms can block PACAP-stimulated pain sensitivity (Missig et al., 2017).

Supporting PACAP roles in pain, several recent studies have shown that PACAP and PAC1 receptor signaling is associated with migraine (Syed et al., 2012; Edvinsson et al., 2018; Akerman and Goadsby, 2015), i.e., PACAP infusions in humans facilitate migraine development. Notably, there are a number of similarities between PACAP and calcitonin gene related peptide (CGRP). CGRP has been implicated in migraine and may also participate in stress-associated behavioral responses (Karsan and Goadsby, 2015), PACAP and CGRP are frequently colocalized in sensory neurons, and PACAP/CGRP signaling mechanisms are similar. The first generation CGRP antagonists have been shown to be effective in treating migraine (Tepper, 2018) but the drugs appeared to induce liver toxicity.

The current CGRP-related therapeutic include biologics targeting the CGRP receptor and 3^{rd}/4^{th} generation CGRP small molecule antagonists that appear to present fewer side effects (some reports of alopecia and hypertension). Current data suggest that the efficacy of CGRP-based therapeutics can be improved. Given the high levels of CGRP/PACAP colocalization and the demonstrated efficacy of CGRP receptor antagonists to ameliorate migraine, comparable or even improved effects may be anticipated for PACAP antagonists.

As proof of principle demonstrating the abilities for PACAP antagonists to inhibit stress-related and pain responses, the infusion of PACAP6-38 into the BNST or amygdala attenuated anxiety-like behavior in chronic stress paradigms (Roman et al., 2014) and blocked nociceptive responses in a sciatic nerve ligation model of chronic pain (Missig et al., 2017). Hence PAC1 receptor antagonists have therapeutic potential in psychopathologies and chronic pain-related disorders. Despite the associations between PACAP signaling and stress-associated behavioral disorders and chronic pain, there are no or few small molecule antagonists to the PACAP receptors for potential therapeutics, including the PACAP-specific PAC1 receptors implicated in stress- and pain-related behaviors and responses. There are a variety of peptide antagonists based on the truncated forms of PACAP. For instance, N-terminally truncated PACAP peptides (i.e., PACAP6-38) can behave as PAC1 receptor antagonists. Further, a deletion variant of maxadilan, a salivary protein (maxd4, M65) identified in sand-fly saliva has been identified as a specific PAC1 receptor antagonist. These peptide and protein antagonists are susceptible to degradation and do not readily cross the blood brain barrier. Hence, these compounds do not appear suitable for candidates for drug development.

From these considerations, small molecule antagonists, especially to the PAC1 receptor are desirable for potential therapeutic applications in stress-related psychopathologies and pain-associated disorders. From current work, PACAP/PAC1 receptor canonical plasma membrane signaling may not be the primary drivers leading to the maladaptation's in stress-related disorders (PTSD) and chronic pain; PACAP/PAC1 receptor internalization and intracellular endosomal signaling, especially ERK activation, appear related in mediating the pathologies. Many putative small molecule antagonists described in the literature have no apparent effects on receptor internalization and signaling. Accordingly, a potent and efficacious small molecule PAC1 receptor antagonist is highly desirable for treatment in stress-related disorders (generalized anxiety disorder, panic disorder, PTSD) and chronic pain-related disorders (migraine, rheumatoid arthritis and related inflammatory pain, musculoskeletal pain). Thus, there remains a need for PACAP antagonists, specifically, small molecule PACAP antagonists.

### SUMMARY OF THE INVENTION

Provided herein are small molecules (e.g., compounds of Formula (I)) that are efficacious in blocking PAC1 receptor endosomal signaling and PACAP-induced stress-related behaviors, and thus have potential therapeutic applications. These small molecule antagonists have the advantage of circumventing the shortcomings of peptidergic receptor antagonists (degradation and inability to cross the blood brain barrier) and may be developed into drugs for therapeutics.

The disclosure entails the development of small molecules that can behave as antagonists to PACAP receptors (e.g. PAC1 receptors), and their methods of use. These molecules may have clinical benefits in ameliorating neurological disorders including, but not limited to, stress-related disorders (e.g. chronic stress, post-traumatic stress disorder (PTSD), panic disorder, anxiety, or general anxiety disorder), eating disorders (e.g. orthorexia, or anorexia nervosa), pain-related disorders (e.g. chronic pain, arthritis (e.g., rheumatoid arthritis), inflammatory pain, musculoskeletal pain, primary headache disorders, or migraines), panic disorders, panic attacks, or addiction (e.g. addiction to a substance (e.g. addiction to cocaine, amphetamines, methamphetamine, methylphenidate, nicotine, alcohol, prescription medication, marijuana, tobacco, food, or an opioid selected from heroin, fentanyl, codeine, hydrocodone, morphine, oxycodone, hydromorphone, and methadone)), neuropathic and emotional pain, and related psycho- and pain-associated pathologies.

Accordingly, in one aspect the disclosure provides compounds of Formula (0):

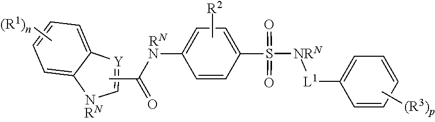

or pharmaceutically acceptable salts, solvates, hydrates, polymorphs, co-crystals, tautomers, isotopologues, stereoisomers, or prodrugs thereof, wherein R¹, R², R³, R^{N}, L¹, Y, n, m, and p are as provided herein. In some embodiments, the compound of Formula (0) is of the formula:

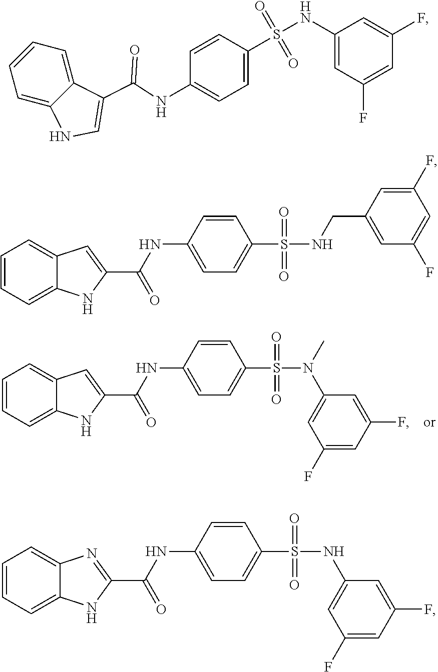

or a salt thereof.

Accordingly, in one aspect the disclosure provides compounds of Formula (I):

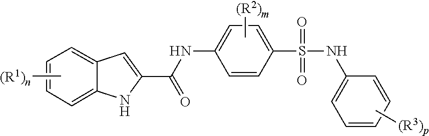

or pharmaceutically acceptable salts, solvates, hydrates, polymorphs, co-crystals, tautomers, isotopologues, stereoisomers, or prodrugs thereof, wherein R¹, R², R³, n, m, and p are as provided herein. In some embodiments, the compound of Formula (I) is of the formula:

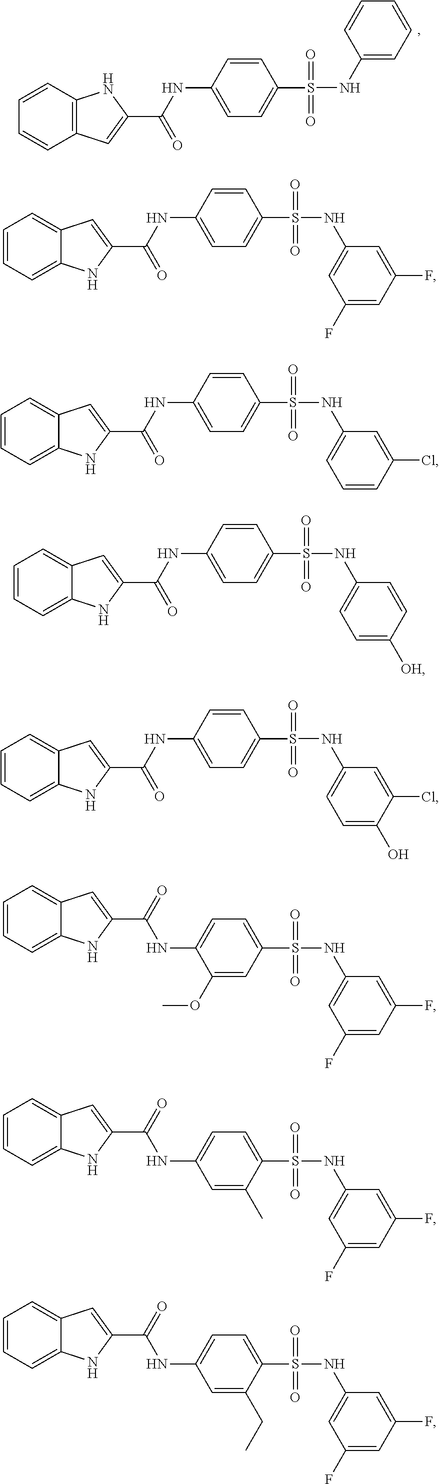

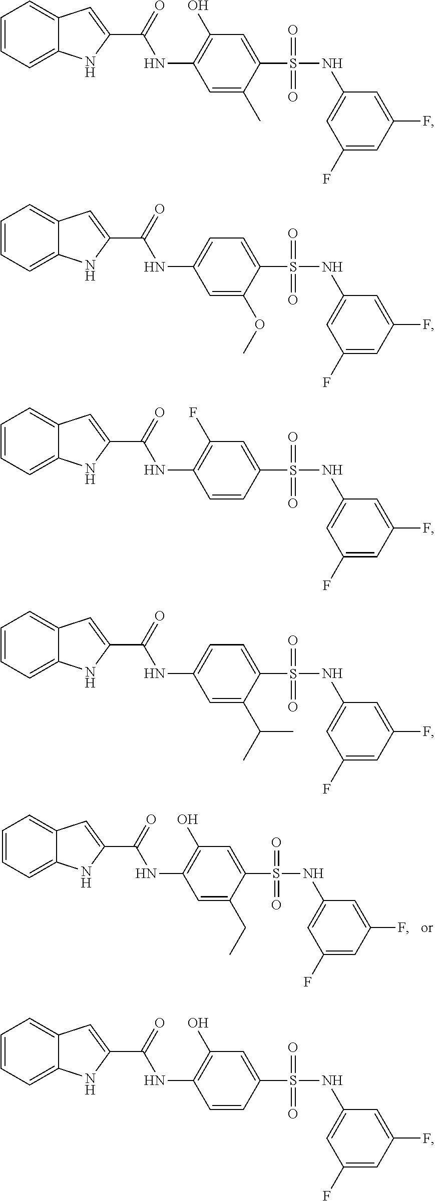 [SMILES: Cc1cc(NC(=O)c2cc3ccccc3[nH]2)c(O)cc1S(=O)(=O)Nc1cc(F)cc(F)c1 ; COc1cc(NC(=O)c2cc3ccccc3[nH]2)ccc1S(=O)(=O)Nc1cc(F)cc(F)c1 ; O=C(Nc1ccc(S(=O)(=O)Nc2cc(F)cc(F)c2)cc1F)c1cc2ccccc2[nH]1 ; CC(C)c1cc(NC(=O)c2cc3ccccc3[nH]2)ccc1S(=O)(=O)Nc1cc(F)cc(F)c1 ; CCc1cc(NC(=O)c2cc3ccccc3[nH]2)c(O)cc1S(=O)(=O)Nc1cc(F)cc(F)c1 ; O=C(Nc1ccc(S(=O)(=O)Nc2cc(F)cc(F)c2)cc1O)c1cc2ccccc2[nH]1]

or a salt thereof. In some embodiments, the compound of Formula (I) is of the formula:

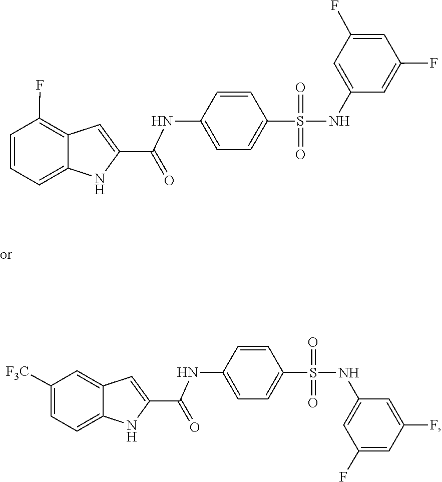 [SMILES: O=C(Nc1ccc(S(=O)(=O)Nc2cc(F)cc(F)c2)cc1)c1cc2c(F)cccc2[nH]1 ; O=C(Nc1ccc(S(=O)(=O)Nc2cc(F)cc(F)c2)cc1)c1cc2cc(C(F)(F)F)ccc2[nH]1]

or salt thereof.

In some aspects, provided herein are pharmaceutical compositions comprising a compound as disclosed herein, or a pharmaceutically acceptable salt thereof, and optionally a pharmaceutically acceptable excipient. In some embodiments, the pharmaceutical composition further comprises an additional pharmaceutical agent.

In other aspects, provided herein are method of treating a subject suffering from or susceptible to a neurological disease, the methods comprising administering to the subject a therapeutically effective amount of a compound of disclosed herein, or pharmaceutically acceptable salt thereof, or a pharmaceutical composition as provided herein. In some embodiments, the neurological disease is a pain-related disorder, stress-related disorder, addiction, a panic disorder, panic attacks, or an eating disorder.

In another aspect, provided herein are methods of treating a subject suffering from or susceptible to a pain-related disease, the method comprising administering to the subject a therapeutically effective amount of a compound of disclosed herein, or pharmaceutically acceptable salt thereof, or a pharmaceutical composition as provided herein. In some embodiments, the pain-related disease is chronic pain, a primary headache disorder (e.g., a migraine), inflammatory pain, musculoskeletal pain, or arthritis (e.g., rheumatoid arthritis).

In an additional aspect, provided herein are methods of treating a subject suffering from or susceptible to a stress-related disease or disorder, the method comprising administering to the subject a therapeutically effective amount of a compound of disclosed herein, or pharmaceutically acceptable salt thereof, or a pharmaceutical composition as provided herein. In some embodiments, the stress-related disorder is chronic stress, posttraumatic stress disorder, (PTSD), an anxiety disorder (e.g., anxiety, generalized anxiety disorder), or a panic disorder.

In one aspect, provided herein are methods of treating a subject suffering from or susceptible to addiction, the method comprising administering to the subject a therapeutically effective amount of a compound of disclosed herein, or pharmaceutically acceptable salt thereof, or a pharmaceutical composition as provided herein. In some embodiments, the addiction is addiction to a substance (e.g., a drug, food, alcohol). In some embodiments, the treatment prevents relapse of the substance abuse. In some embodiments, the treatment lowers the incidence of relapse of the substance abuse. In some embodiments, the relapse is stress-induced relapse.

In another aspect, provided herein are methods of treating a subject suffering from or susceptible to an eating disorder, the method comprising administering to the subject a therapeutically effective amount of a compound of disclosed herein, or pharmaceutically acceptable salt thereof, or a pharmaceutical composition as provided herein. In some embodiments, the eating disorder is orthorexia or anorexia.

In one aspect, provided herein are methods of treating a subject identified as in need of treatment for a pain-related disease or disorder, the method comprising administering to the subject a therapeutically effective amount of a compound of disclosed herein, or pharmaceutically acceptable salt thereof, or a pharmaceutical composition as provided herein.

In another aspect, provided herein are methods of treating a subject identified as in need of treatment for a stress-related disorder, the method comprising administering to the subject a therapeutically effective amount of a compound of disclosed herein, or pharmaceutically acceptable salt thereof, or a pharmaceutical composition as provided herein.

In an additional aspect, provided herein are methods of treating a subject identified as in need of treatment for a panic disorder, the method comprising administering to the subject a therapeutically effective amount of a compound of disclosed herein, or pharmaceutically acceptable salt thereof, or a pharmaceutical composition as provided herein.

In one aspect, provided herein are methods of treating a subject identified as in need of treatment for addiction, the method comprising administering to the subject a therapeutically effective amount of a compound of disclosed herein, or pharmaceutically acceptable salt thereof, or a pharmaceutical composition as provided herein.

In another aspect, provided herein are methods of treating a subject identified as in need of treatment for an eating disorder, the method comprising administering to the subject a therapeutically effective amount of a compound of disclosed herein, or pharmaceutically acceptable salt thereof, or a pharmaceutical composition as provided herein.

In some aspects, the methods provided herein further comprise administering an additional pharmaceutical agent to the subject.

In one aspect, provided herein are kits comprising: a compound of disclosed herein, or pharmaceutically acceptable salt thereof, or a pharmaceutical composition as provided herein.; and instructions for administering to a subject or contacting a biological sample with the compound, or composition.

In some aspects, provided herein are methods for modulating a pituitary adenylate cyclase activating polypeptide (PACAP) receptor, comprising contacting a PAC1 receptor with a compound of disclosed herein, or pharmaceutically acceptable salt thereof, or a pharmaceutical composition as provided herein.

In an additional aspect, provided herein are methods for modulating a PAC1 receptor, comprising contacting a PAC1 receptor with a compound of disclosed herein, or pharmaceutically acceptable salt thereof, or a pharmaceutical composition as provided herein.

In one aspect, provided herein are methods of treating a disease or disorder in a subject by modulating a pituitary adenylate cyclase activating polypeptide (PACAP) receptor, the method comprising administering to the subject a therapeutically effective amount of a compound of disclosed herein, or pharmaceutically acceptable salt thereof, or a pharmaceutical composition as provided herein.

In some aspects, provided herein are methods of treating a disease or disorder in a subject by modulating a PAC1 receptor, the method comprising administering to the subject a therapeutically effective amount of a compound of disclosed herein, or pharmaceutically acceptable salt thereof, or a pharmaceutical composition as provided herein.

The details of one or more embodiments of the invention are set forth herein. Other features, objects, and advantages of the invention will be apparent from the Detailed Description, Examples, figures, and Claims.

### Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987. The disclosure is not intended to be limited in any manner by the exemplary listing of substituents described herein.

Compounds described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, NY, 1962); and Wilen, Tables of Resolving Agents and Optical Resolutions p. 268 (E.L. Eliel, Ed., Univ. of Notre Dame Press, Notre Dame, IN 1972). The disclosure additionally encompasses compounds described herein as individual isomers substantially free of other isomers, and alternatively, as mixtures of various isomers. While compounds may be depicted as racemic or as one or more diastereoisomers, enantiomers, or other isomers, all such racemic, diastereoisomer, enantiomer, or other isomer forms of that depicted are included in the present disclosure.

In a formula, [symbol] is a single bond where the stereochemistry of the moieties immediately attached thereto is not specified but can be any stereochemistry (e.g., D or L).

Unless otherwise stated, structures depicted herein are also meant to include compounds that differ only in the presence of one or more isotopically enriched atoms. For example, compounds having the present structures except for the replacement of hydrogen by deuterium or tritium, replacement of ¹⁹F with ¹⁸F, or the replacement of ¹²C with ¹³C or ¹⁴C are within the scope of the disclosure. Such compounds are useful, for example, as analytical tools or probes in biological assays.

The term “alkyl” refers to a radical of a straight-chain or branched saturated hydrocarbon group having from 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), propyl (C₃) (e.g., n-propyl, isopropyl), butyl (C₄) (e.g., n-butyl, tert-butyl, sec-butyl, iso-butyl), pentyl (C₅) (e.g., n-pentyl, 3-pentanyl, amyl, neopentyl, 3-methyl-2-butanyl, tertiary amyl), and hexyl (C₆) (e.g., n-hexyl). Additional examples of alkyl groups include n-heptyl (C₇), n-octyl (C₃), and the like. Unless otherwise specified, each instance of an alkyl group is independently unsubstituted (an “unsubstituted alkyl”) or substituted (a “substituted alkyl”) with one or more substituents (e.g., halogen, such as F). In certain embodiments, the alkyl group is an unsubstituted C₁₋₁₀ alkyl (such as unsubstituted C₁₋₆ alkyl, e.g., —CH₃ (Me), unsubstituted ethyl (Et), unsubstituted propyl (Pr, e.g., unsubstituted n-propyl (n-Pr), unsubstituted isopropyl (i-Pr)), unsubstituted butyl (Bu, e.g., unsubstituted n-butyl (n-Bu), unsubstituted tert-butyl (tert-Bu or t-Bu), unsubstituted sec-butyl (sec-Bu), unsubstituted isobutyl (i-Bu)). In certain embodiments, the alkyl group is a substituted C₁₋₁₀ alkyl (such as substituted C₁₋₆ alkyl, e.g., —CF₃, Bn). In certain embodiments, the alkyl group is an unsubstituted C₁₋₆ alkyl (e.g., —CH₃ (Me), unsubstituted ethyl (Et), unsubstituted propyl (Pr, e.g., unsubstituted n-propyl (n-Pr), unsubstituted isopropyl (i-Pr)), unsubstituted butyl (Bu, e.g., unsubstituted n-butyl (n-Bu), unsubstituted tert-butyl (tert-Bu or t-Bu), unsubstituted sec-butyl (sec-Bu), unsubstituted isobutyl (i-Bu)). In certain embodiments, the alkyl group is a substituted C₁₋₆ alkyl (e.g., —CF₃, Bn).

The term “halo” or “halogen” refers to fluorine (fluoro, —F), chlorine (chloro, —Cl), bromine (bromo, —Br), or iodine (iodo, —I).

The term “hydroxyl” or “hydroxy” refers to the group —OH.

The term “alkoxy” refers to a group —O-(substituted or unsubstituted C₁₋₆ alkyl).

The term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, Berge et al., describe pharmaceutically acceptable salts in detail in J. Pharmaceutical Sciences, 1977, 66, 1-19, incorporated herein by reference. Pharmaceutically acceptable salts of the compounds of this disclosure include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid, and perchloric acid or with organic acids such as acetic acid, oxalic acid, maleic acid, tartaric acid, citric acid, succinic acid, or malonic acid. These salts could be formed by protonation of the compound with the requisite acid, or by other methods known in the art such as ion exchange or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N⁺(C₁₋₄ alkyl)₄⁻ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

The term “solvate” refers to forms of the compound that are associated with a solvent, usually by a solvolysis reaction. This physical association may include hydrogen bonding. Conventional solvents include water, methanol, ethanol, acetic acid, DMSO, THF, diethyl ether, and the like. The compounds of Formula (I) may be prepared, e.g., in crystalline form, and may be solvated. Suitable solvates include pharmaceutically acceptable solvates and further include both stoichiometric solvates and non-stoichiometric solvates. In certain instances, the solvate will be capable of isolation, for example, when one or more solvent molecules are incorporated in the crystal lattice of a crystalline solid. “Solvate” encompasses both solution-phase and isolable solvates. Representative solvates include hydrates, ethanolates, and methanolates.

The term “hydrate” refers to a compound that is associated with water. Typically, the number of the water molecules contained in a hydrate of a compound is in a definite ratio to the number of the compound molecules in the hydrate. Therefore, a hydrate of a compound may be represented, for example, by the general formula R·x H₂O, wherein R is the compound and wherein x is a number greater than 0. A given compound may form more than one type of hydrates, including, e.g., monohydrates (x is 1), lower hydrates (x is a number greater than 0 and smaller than 1, e.g., hemihydrates (R·0.5 H₂O)), and polyhydrates (x is a number greater than 1, e.g., dihydrates (R·2 H₂O) and hexahydrates (R·6 H₂O)).

The term “tautomers” refer to compounds that are interchangeable forms of a particular compound structure, and that vary in the displacement of hydrogen atoms and electrons. Thus, two structures may be in equilibrium through the movement of π electrons and an atom (usually H). For example, enols and ketones are tautomers because they are rapidly interconverted by treatment with either acid or base. Another example of tautomerism is the aci- and nitro-forms of phenylnitromethane, that are likewise formed by treatment with acid or base.

Tautomeric forms may be relevant to the attainment of the optimal chemical reactivity and biological activity of a compound of interest.

It is also to be understood that compounds that have the same molecular formula but differ in the nature or sequence of bonding of their atoms or the arrangement of their atoms in space are termed “isomers.” Isomers that differ in the arrangement of their atoms in space are termed “stereoisomers.”

Stereoisomers that are not mirror images of one another are termed “diastereomers” and those that are non-superimposable mirror images of each other are termed “enantiomers.” When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture.”

The term “isotopologue” refers to compounds that differ only in their isotopic composition. Unless otherwise stated, structures depicted herein are also meant to include compounds that differ only in the presence of one or more isotopically enriched atoms. For example, compounds having the present structures except for the replacement of hydrogen by deuterium or tritium, replacement of ¹⁹F with ¹⁸F, or the replacement of ¹²C with ¹³C or ¹⁴C are within the scope of the disclosure. Such compounds are useful, for example, as analytical tools or probes in biological assays.

The term “polymorphs” refers to a crystalline form of a compound (or a salt, hydrate, or solvate thereof) in a particular crystal packing arrangement. Polymorphs have the same elemental composition. Different crystalline forms usually have different X-ray diffraction patterns, infrared spectra, melting points, density, hardness, crystal shape, optical and electrical properties, stability, and solubility. Recrystallization solvent, rate of crystallization, storage temperature, and other factors may cause one crystal form to dominate. Various polymorphs of a compound can be prepared by crystallization under different conditions.

The term “prodrugs” refer to compounds, including derivatives of the compounds of Formula (I), which have cleavable groups and become by solvolysis or under physiological conditions the compounds of Formula (I), which are pharmaceutically active in vivo. Such examples include, but are not limited to, ester derivatives and the like. Other derivatives of the compounds of this disclosure have activity in both their acid and acid derivative forms, but in the acid sensitive form often offers advantages of solubility, tissue compatibility, or delayed release in the mammalian organism (see, Bundgaard, H., Design of Prodrugs, pp. 7-9, 21-24, Elsevier, Amsterdam 1985). Prodrugs include acid derivatives well known to practitioners of the art, such as, for example, esters prepared by reaction of the parent acid with a suitable alcohol, or amides prepared by reaction of the parent acid compound with a substituted or unsubstituted amine, or acid anhydrides, or mixed anhydrides. Simple aliphatic or aromatic esters, amides, and anhydrides derived from acidic groups pendant on the compounds of this disclosure are particular prodrugs. In some cases it is desirable to prepare double ester type prodrugs such as (acyloxy)alkyl esters or ((alkoxycarbonyl)oxy)alkylesters. C₁-C₈ alkyl, C₂-C₈ alkenyl, C₂-C₈ alkynyl, aryl, C₇-C₁₂ substituted aryl, and C₇-C₁₂ arylalkyl esters.

A “subject” to which administration is contemplated includes, but is not limited to, humans (i.e., a male or female of any age group, e.g., a pediatric subject (e.g., infant, child, adolescent) or adult subject (e.g., young adult, middle-aged adult, or senior adult)) and/or other non-human animals, for example, mammals (e.g., primates (e.g., cynomolgus monkeys, rhesus monkeys); commercially relevant mammals such as cattle, pigs, horses, sheep, goats, cats, and/or dogs) and birds (e.g., commercially relevant birds such as chickens, ducks, geese, and/or turkeys). In certain embodiments, the animal is a mammal. The animal may be a male or female and at any stage of development. A non-human animal may be a transgenic animal.

The terms “administer,” “administering,” or “administration,” refers to implanting, absorbing, ingesting, injecting, inhaling, or otherwise introducing an inventive compound, or a pharmaceutical composition thereof.

The terms “treatment,” “treat,” and “treating” refer to reversing, alleviating, or inhibiting the progress of a “pathological condition” (e.g., a disease, disorder, or condition, or one or more signs or symptoms thereof) described herein. In some embodiments, treatment may be administered after one or more signs or symptoms have developed or have been observed. In other embodiments, treatment may be administered in the absence of signs or symptoms of the disease or condition. For example, treatment may be administered to a susceptible individual prior to the onset of symptoms (e.g., in light of a history of symptoms and/or in light of genetic or other susceptibility factors). Treatment may also be continued after symptoms have resolved, for example, to delay or prevent recurrence.

The terms “condition,” “disease,” and “disorder” are used interchangeably.

The term “modulate” refers to an increase or decrease of a biological event (e.g., the amount of receptor internalization, or ERK activation) in response to contact with a compound of the disclosure, to a level that is higher or lower than an initial or other appropriate reference level, which may, for example, be a baseline level of a target. Examples of modulating agents include, but are not limited to agonists and antagonists.

The term “receptor” refers to any protein molecule that receives a signal from outside or inside a cell. In general, receptors are membrane bound proteins. A receptor may be a peripheral membrane protein, an integral membrane protein, or any protein that interacts with the cellular membrane. A receptor may be comprised of a single protein or a complex of two or more proteins. Receptors induce a type of cellular response when a chemical signal or molecule binds to the receptor. A receptor may also refer to any drug target, such as an enzyme, transporter, or ion channel that is the target of a drug. Examples of receptors include, but are not limited to, ionotropic receptors, PACAP receptors (e.g. PAC1), G-protein coupled receptors, receptor tyrosine kinases, and nuclear receptors.

The term “antagonist” refers to an agent that (i) decreases or suppresses one or more effects of another agent; and/or (ii) decreases or suppresses one or more biological events. In some embodiments, an antagonist may reduce level and/or activity of one or more agents that it targets. In various embodiments, antagonists may be or include agents of various chemical class including, for example, small molecules, acyl hydrazides, polypeptides, nucleic acids, carbohydrates, lipids, metals, and/or other entity that shows the relevant antagonistic activity. An antagonist may be direct (in which case it exerts its influence directly upon its target) or indirect (in which case it exerts its influence by other than binding to its target; e.g., by interacting with a regulator of the target, for example so that level or activity of the target is altered). In some embodiments, an antagonist may be a receptor antagonist, e.g., a receptor ligand or drug that blocks or dampens a biological response by binding to and blocking a receptor rather than activating it like an agonist.

The term “agonist” refers to an agent that (i) increases or induces one or more effects of another agent; and/or (ii) increases or induces one or more biological events. In some embodiments, an agonist may increase level and/or activity of one or more agents that it targets. In various embodiments, agonists may be or include agents of various chemical class including, for example, small molecules, polypeptides, nucleic acids, carbohydrates, lipids, metals, and/or other entity that shows the relevant agonistic activity. An agonist may be direct (in which case it exerts its influence directly upon its target) or indirect (in which case it exerts its influence by other than binding to its target; e.g., by interacting with a regulator of the target, for example so that level or activity of the target is altered). A partial agonist can act as a competitive antagonist in the presence of a full agonist, as it competes with the full agonist to interact with its target and/or a regulator thereof, thereby producing (i) a decrease in one or more effects of another agent, and/or (ii) a decrease in one or more biological events, as compared to that observed with the full agonist alone.

The term “inhibit” or “inhibition” in the context of modulating level (e.g., expression and/or activity) of a target (e.g., a PACAP receptor (e.g. PAC1)) is not limited to only total inhibition. Thus, in some embodiments, partial inhibition or relative reduction is included within the scope of the term “inhibition.” In some embodiments, the term refers to a reduction of the level (e.g., expression, internalization, and/or activity) of a target (e.g., a PACAP receptor (e.g. PAC1) to a level that is reproducibly and/or statistically significantly lower than an initial or other appropriate reference level, which may, for example, be a baseline level of a target. In some embodiments, the term refers to a reduction of the level (e.g., expression and/or activity) of a target to a level that is less than 75%, less than 50%, less than 40%, less than 30%, less than 25%, less than 20%, less than 10%, less than 9%, less than 8%, less than 7%, less than 6%, less than 5%, less than 4%, less than 3%, less than 2%, less than 1%, less than 0.5%, less than 0.1%, less than 0.01%, less than 0.001%, or less than 0.0001% of an initial level, which may, for example, be a baseline level of a target.

An “effective amount” of a compound of Formula (I), refers to an amount sufficient to elicit the desired biological response, i.e., treating the condition. As will be appreciated by those of ordinary skill in this art, the effective amount of a compound of Formula (I) may vary depending on such factors as the desired biological endpoint, the pharmacokinetics of the compound, the condition being treated, the mode of administration, and the age and health of the subject. An effective amount encompasses therapeutic and prophylactic treatment. For example, in treating cancer, an effective amount of an inventive compound may reduce the tumor burden or stop the growth or spread of a tumor.

A “therapeutically effective amount” of a compound of Formula (I), is an amount sufficient to provide a therapeutic benefit in the treatment of a condition or to delay or minimize one or more symptoms associated with the condition. A therapeutically effective amount of a compound means an amount of therapeutic agent, alone or in combination with other therapies, which provides a therapeutic benefit in the treatment of the condition. The term “therapeutically effective amount” can encompass an amount that improves overall therapy, reduces, or avoids symptoms or causes of the condition, or enhances the therapeutic efficacy of another therapeutic agent.

A “prophylactically effective amount” of a compound of Formula (I), is an amount sufficient to prevent a condition, or one or more symptoms associated with the condition or prevent its recurrence. A prophylactically effective amount of a compound means an amount of a therapeutic agent, alone or in combination with other agents, which provides a prophylactic benefit in the prevention of the condition. The term “prophylactically effective amount” can encompass an amount that improves overall prophylaxis or enhances the prophylactic efficacy of another prophylactic agent.

The term “neurological disorder” refers to any disease or disorder of the nervous system, including diseases that involve the central nervous system (brain, brainstem and cerebellum), the peripheral nervous system (including cranial nerves), and the autonomic nervous system (parts of which are located in both central and peripheral nervous system). Examples of neurological diseases include, but are not limited to, stress-related disorders, pain-related disorders, panic disorders, panic attacks, eating disorders, and addiction. Stress-related disorders include, but are not limited to chronic stress, post-traumatic stress disorder (PTSD), panic disorder, anxiety, and general anxiety disorder. Pain-related disorders include, but are not limited to chronic pain, primary headache disorders, arthritis (e.g., rheumatoid arthritis), inflammatory pain, musculoskeletal pain, and migraines. Eating disorders include, but are not limited to orthorexia, and anorexia nervosa. Addictions include, but are not limited to addiction to a substance (e.g. cocaine, amphetamines, methamphetamine, methylphenidate, nicotine, alcohol, prescription medication, marijuana, tobacco, food, or an opioid selected from heroin, fentanyl, codeine, hydrocodone, morphine, oxycodone, hydromorphone, and methadone).

The term “biological sample” refers to any sample including tissue samples (such as tissue sections and needle biopsies of a tissue); cell samples (e.g., cytological smears (such as Pap or blood smears) or samples of cells obtained by microdissection); samples of whole organisms (such as samples of yeasts or bacteria); or cell fractions, fragments or organelles (such as obtained by lysing cells and separating the components thereof by centrifugation or otherwise). Other examples of biological samples include blood, serum, urine, semen, fecal matter, cerebrospinal fluid, interstitial fluid, mucus, tears, sweat, pus, biopsied tissue (e.g., obtained by a surgical biopsy or needle biopsy), nipple aspirates, milk, vaginal fluid, saliva, swabs (such as buccal swabs), or any material containing biomolecules that is derived from a first biological sample. Biological samples also include those biological samples that are transgenic, such as a transgenic oocyte, sperm cell, blastocyst, embryo, fetus, donor cell, or cell nucleus.

### BRIEF DESCRIPTION OF THE DRAWINGS

FIG. 1 depicts Western blot data for inhibition of PACAP-stimulated ERK activation by compounds of the disclosure.

FIG. 2 depicts the dose-dependent inhibition of PACAP-stimulated ERK activation by compounds of the disclosure.

FIG. 3 depicts that PAC1 receptor antagonist RJB-145 blocks PACAP-induced signaling and anxiety-like behavioral responses. FIG. 3A shows the PACAP-induced calcium influx. Using HEK PAC1R stable cell lines, RJB-145 attenuated 20 nM PACAP-induced calcium flux (filled circles) and ERK activation (open circles) with calculated IC₅₀ of 8.5 and 9 uM, respectively. 20 μM RJB-145 blocked PACAP-stimulated ERK activation nearly 75%. Data represent mean +/− SEM; n=3−4 wells/group. FIG. 3B shows in vivo assessments of the antagonist, vehicle, RJB-145 (20 μM/0.5 μL), PACAP (1 μg/μl; 0.5 μl infusion) or PACAP+RJB−145 were infused bilaterally into the BNST (bed nucleus of the stria terminalis, a brain nuclei for stress responses) of rats for anxiety-like measurements on an elevated plus maze (EPM; 5 min per trial). Anxiety ridden rats avoid the open arms of the maze and prefer traveling into the closed arms. RJB-145 infusions alone had no behavioral effects compared to vehicle. Consistent with previous work, PACAP instilled anxiety-like responses as shown by rats avoiding the open arms (decreased open arm times in the figure). RJB145 was able to completely antagonize the PACAP-induced anxiety-like responses to control vehicle levels. Data represent mean +/− SEM; n=8-10 animals/group; **, statistically different from vehicle, RJB145 and PACAP+RJB145 at p<0.01.

### DETAILED DESCRIPTION OF CERTAIN EMBODIMENTS OF THE INVENTION

From previous work (Beebe et al., 2008) acyl hydrazide compounds appear to have the potential to antagonize PACAP plasma membrane calcium signaling; however, while these candidate compounds can attenuate PAC1 receptor mediated calcium flux, they have modest effects in blocking PAC1 receptor internalization and endosomal ERK activation. These results are not surprising given the bias agonism/antagonism states (how different ligands can cause G protein coupled receptors to adopt altered conformational states resulting in differential signaling responses) that are now understood to exist for G protein coupled receptors.

From background and signaling data, we have posited that for therapeutic potential, PAC1 receptor antagonists should demonstrate efficacy in blunting both plasma membrane (i.e., calcium flux) and endosomal ERK signaling. In recent work, acyl hydrazide analogs were prepared and from these, several new compounds have shown efficacy in inhibiting PACAP/PAC1 receptor-stimulated plasma membrane signaling, internalization and endosomal ERK activation in a stable human PAC1 receptor-HEK cell line. The novel derivatives differed from the original lead compound by modification of the substituents present on the N-benzyl ring. Specifically, the inclusion of electron withdrawing groups on the N-benzyl ring appeared to be relevant for blocking receptor internalization and endosomal ERK activation.

One derivative (JF-214) bearing a 3,5-difluorobenzyl group:

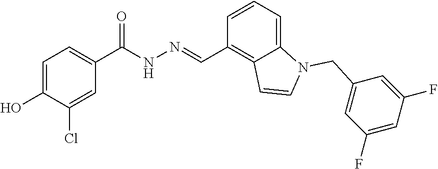 [SMILES: O=C(N/N=C/c1cccc2c1ccn2Cc1cc(F)cc(F)c1)c1ccc(O)c(Cl)c1]

showed particular efficacy—the new heteroatoms could take advantage of a hydrogen bonding interactions within the binding pocket, or the electron deficient aryl ring may be involved in π-stacking or cation-π interactions within the receptor. The presence of fluorine or other electron withdrawing groups at the 3 and 5 positions of the aromatic ring appeared central to the drug design. It was discovered that, JF-214: 1) blocked PACAP-stimulated PAC1 receptor plasma membrane calcium signaling; 2) blocked PAC1 receptor internalization and endosomal ERK signaling; 3) acted synergistically with extrahelical glucagon receptor (GCGR) antagonist MK-0893 to inhibit PACAP/PAC1 receptormediated ERK activation; and 4) demonstrated efficacy in blocking PACAP-induced anxietyrelated behaviors in vivo. JF-214 and related compounds have been submitted for patent protection (PCT publication number WO/2020/168068, U.S. Ser. No. 17/429,843).

Computer modeling and virtual screening were carried out to identify new scaffolds capable of behaving as PAC1 receptor antagonists. Despite the lack of inactive or antagonist-bound PAC1 structural information, the hypothesis of JF-214 interactions with the PAC1 receptor helped to build four accurate protein models. With a unique ensemble docking protocol, 3 millions of small molecules with similar chemical properties to JF-214 were tested in the orthosteric pocket of PAC1. The top compounds with high binding scores were selected for experimental validation and are disclosed herein.

The present disclosure provides compounds that act as antagonists for a PACAP-receptor (e.g. PAC1), and pharmaceutical compositions thereof, for the prevention and treatment of a disease or disorder in a subject. In certain embodiments, the compounds act as antagonists to PACAP receptors (e.g. PAC1). In certain embodiments, the compounds are irreversible antagonist for PACAP receptors (e.g. PAC1). In certain embodiments, the compounds are reversible antagonist for PACAP receptors (e.g. PAC1). The present disclosure further provides methods of using the compounds described herein, e.g., as biological probes to study the antagonism of PACAP receptors (e.g. PAC1), and as therapeutics, e.g., in the prevention and treatment of diseases or disorders associated with PACAP receptor activity or internalization, or ERK activation. In certain embodiments, the diseases or disorders include, but are not limited to, neurological disorders, e.g. stress-related disorders (e.g. chronic stress, post-traumatic stress disorder (PTSD), panic disorder, anxiety, or general anxiety disorder), eating disorders (e.g. orthorexia, or anorexia nervosa), pain-related disorders (e.g. chronic pain, arthritis (e.g., rheumatoid arthritis), inflammatory pain, musculoskeletal pain, primary headache disorders, or migraines), panic disorders, panic attacks, or addiction (e.g. addiction to a substance (e.g. addiction to cocaine, amphetamines, methamphetamine, methylphenidate, nicotine, alcohol, prescription medication, marijuana, tobacco, food, or an opioid selected from heroin, fentanyl, codeine, hydrocodone, morphine, oxycodone, hydromorphone, and methadone)).

### Compounds

In one aspect, provided herein is a compound of Formula (0):

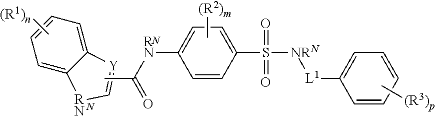

or a pharmaceutically acceptable salt thereof, wherein:
   each R¹ is independently substituted or unsubstituted C₁₋₆ alkyl, halogen, hydroxy, substituted or unsubstituted C₁₋₆ alkoxy, —NR^{a}R^{a}, —C(═O)R^{a}, —C(═O)OR^{a}, —NR^{a}C(═O)R^{a}, —C(═O)N(R^{a})₂, —CN, or —NO₂;
   each R² is independently substituted or unsubstituted C₁₋₆ alkyl, halogen, hydroxy, substituted or unsubstituted C₁₋₆ alkoxy, —NR^{a}R^{a}, —C(═O)R^{a}, —C(═O)OR^{a}, —NR^{a}C(═O)R^{a}, —C(═O)N(R^{a})₂, —CN, or —NO₂;
   each R³ is independently halogen, hydroxy, —NR^{a}R^{a}, —C(═O)R^{a}, —C(═O)OR^{a}, —CN, or —NO₂;
   each R^{N} is independently hydrogen or substituted or unsubstituted C₁₋₆ alkyl;
   each R^{a} is independently H or substituted or unsubstituted C₁₋₆ alkyl;
   Y is —CH—, —N—, or

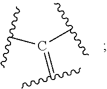 [SMILES: *C(*)=*]

L¹ is a bond or substituted or unsubstituted

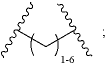

n is 0, 1, 2, 3, or 4;
m is 0, 1, 2, 3, or 4; and
p is 0, 1, 2, 3, 4, or 5.

In some embodiments, the compound of Formula (0) is of Formula (0-A):

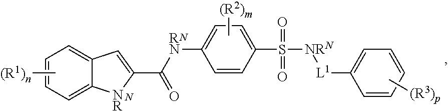

or a pharmaceutically acceptable salt thereof.

In some embodiments, the compound of Formula (0) is of Formula (0-B):

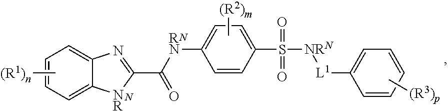

or a pharmaceutically acceptable salt thereof.

In some embodiments, the compound of Formula (0) is of Formula (0-C):

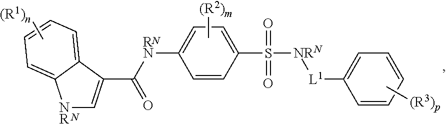

or a pharmaceutically acceptable salt thereof.

In some embodiments, the compound of Formula (0) is of Formula (0-D):

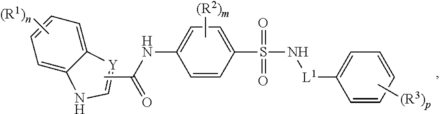

or a pharmaceutically acceptable salt thereof.

In some embodiments, the compound of Formula (0) is of Formula (0-E):

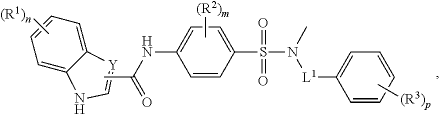

or a pharmaceutically acceptable salt thereof.

In some embodiments, each R^{N} is independently hydrogen or substituted or unsubstituted C₁₋₆ alkyl. In some embodiments, each R^{N} is hydrogen. In some embodiments, each R^{N} is C₁₋₆ alkyl. In some embodiments, each R^{N} is methyl. In some embodiments, some instances of R^{N} are hydrogen and some instances are methyl. In some embodiments, two instances of R^{N} are hydrogen and one instance of R^{N} is methyl.

In some embodiments, Y is —CH—, —N—, or

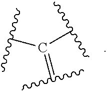 [SMILES: *C(*)=*]

In some embodiments, Y is —CH—. In certain embodiments, Y is —N—. In some embodiments, Y is

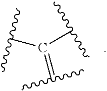 [SMILES: *C(*)=*]

In some embodiments, L¹ is a bond or substituted or unsubstituted

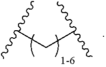

In some embodiments, L¹ is a bond. In certain embodiments, L¹ is substituted or unsubstituted

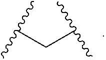 [SMILES: *C*]

In certain embodiments, L¹ is unsubstituted

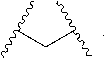 [SMILES: *C*]

In certain embodiments, L¹ is substituted or unsubstituted

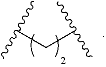

In certain embodiments, L¹ is substituted or unsubstituted

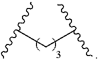

In one aspect of the present disclosure, provided are compounds of Formula (I):

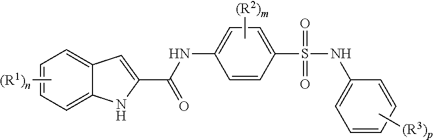

or pharmaceutically acceptable salts, solvates, hydrates, polymorphs, co-crystals, tautomers, isotopologues, stereoisomers, or prodrugs thereof, wherein:
   each R¹ is independently substituted or unsubstituted C₁₋₆ alkyl, halogen, hydroxy, substituted or unsubstituted C₁₋₆ alkoxy, —NR^{a}R^{a}, —C(═O)R^{a}, —C(═O)OR^{a}, —NR^{a}C(═O)R^{a}, —C(═O)N(R^{a})₂, —CN, or —NO₂;
   each R² is independently substituted or unsubstituted C₁₋₆ alkyl, halogen, hydroxy, substituted or unsubstituted C₁₋₆ alkoxy, —NR^{a}R^{a}, —C(═O)R^{a}, —C(═O)OR^{a}, —NR^{a}C(═O)R^{a}, —C(═O)N(R^{a})₂, —CN, or —NO₂;
   each R³ is independently halogen, hydroxy, —NR^{a}R^{a}, —C(═O)R^{a}, —C(═O)OR^{a}, —CN, or —NO₂;
   each R^{a} is independently H or substituted or unsubstituted C₁₋₆ alkyl;
   n is 0, 1, 2, 3, or 4;
   m is 0, 1, 2, 3, or 4; and
   p is 0, 1, 2, 3, 4, or 5.

In some embodiments, each R² is independently substituted or unsubstituted C₁₋₆ alkyl, halogen, hydroxy, substituted or unsubstituted C₁₋₆ alkoxy, —C(═O)R^{a}, —C(═O)OR^{a}, —NR^{a}C(═O)R^{a}, —C(═O)N(R^{a})₂, —CN, or —NO₂; each R³ is independently halogen, hydroxy, —C(═O)R^{a}, —C(═O)OR^{a}, —CN, or —NO₂; each R^{a} is independently H or substituted or unsubstituted C₁₋₆ alkyl; n is 0; m is 0, 1, 2, 3, or 4; and p is 0, 1, 2, 3, or 4.

In some embodiments, each R² is independently substituted or unsubstituted C₁₋₆ alkyl, halogen, hydroxy, or substituted or unsubstituted C₁₋₆ alkoxy; each R³ is independently halogen, —C(═O)R^{a}, —C(═O)OR^{a}, —CN, or —NO₂; each R^{a} is independently H or substituted or unsubstituted C₁₋₆ alkyl; n is 0; m is 0, 1, 2, 3, or 4; and p is 0, 1, 2, 3, or 4.

As provided herein, each R¹ is independently substituted or unsubstituted C₁₋₆ alkyl, halogen, hydroxy, substituted or unsubstituted C₁₋₆ alkoxy, —NR^{a}R^{a}, —C(═O)R^{a}, —C(═O)OR^{a}, —NR^{a}C(═O)R^{a}, —C(═O)N(R^{a})₂, —CN, or —NO₂. In some embodiments, R¹ is substituted or unsubstituted C₁₋₆ alkyl, halogen, hydroxy, substituted or unsubstituted C₁₋₆ alkoxy, —NR^{a}R^{a}, —C(═O)R^{a}, —C(═O)OR^{a}, —NR^{a}C(═O)R^{a}, —C(═O)N(R^{a})₂, —CN, or —NO₂. In some embodiments, R¹ is substituted or unsubstituted C₁₋₆ alkyl. In some embodiments, R¹ is unsubstituted C₁₋₆ alkyl. In certain embodiments, R¹ is methyl. In some embodiments, R¹ is ethyl. In some embodiments, R¹ is propyl. In some embodiments, R¹ is isopropyl. In certain embodiments, R¹ is butyl. In some embodiments, R¹ is tert-butyl. In some embodiments, R¹ is substituted C₁₋₆ alkyl. In certain embodiments, R¹ is substituted methyl (e.g., —CF₃). In some embodiments, R¹ is —CF₃. In some embodiments, R¹ is substituted ethyl. In some embodiments, R¹ is substituted propyl. In some embodiments, R¹ is substituted isopropyl. In certain embodiments, R¹ is substituted butyl. In some embodiments, R¹ is substituted or unsubstituted C₁₋₆ alkoxy. In some embodiments, R¹ is unsubstituted C₁₋₆ alkoxy. In certain embodiments, R¹ is methoxy. In some embodiments, R¹ is ethoxy. In some embodiments, R¹ is propoxy. In certain embodiments, R¹ is isopropoxy. In some embodiments, R¹ is butoxy. In certain embodiments, R¹ is tert-butoxy. In some embodiments, R¹ is substituted C₁₋₆ alkoxy. In certain embodiments, R¹ is halogen. In some embodiments, R¹ is chloro, fluoro, bromo, or iodo. In certain embodiments, R¹ is chloro. In some embodiments, R¹ is fluoro. In certain embodiments, R¹ is bromo. In some embodiments, R¹ is iodo. In some embodiments, R¹ is hydroxy. In certain embodiments, R¹ is —NR^{a}R^{a}. In certain embodiments, R¹ is —N(CH₃)₂. In certain embodiments, R¹ is —NH(CH₃). In certain embodiments, R¹ is —NH₂. In certain embodiments, R¹ is —C(═O)R^{a}. In certain embodiments, R¹ is —C(═O)H. In certain embodiments, R¹ is —C(═O)Me. In certain embodiments, R¹ is —C(═O)OR^{a}. In certain embodiments, R¹ is —C(═O)OH. In certain embodiments, R¹ is —C(═O)OMe. In certain embodiments, R¹ is —NR^{a}C(═O)R^{a}. In certain embodiments, R¹ is —NHC(═O)H. In certain embodiments, R¹ is —NHC(═O)Me. In certain embodiments, R¹ is —N(Me)C(═O)H. In certain embodiments, R¹ is —N(Me)C(═O)Me. In certain embodiments, R¹ is —C(═O)N(R^{a})₂. In certain embodiments, R¹ is —C(═O)NH₂. In certain embodiments, R¹ is —C(═O)NHMe. In certain embodiments, R¹ is —C(═O)N(Me)₂. In certain embodiments, R¹ is —CN.

As provided herein, each R² is independently substituted or unsubstituted C₁₋₆ alkyl, halogen, hydroxy, substituted or unsubstituted C₁₋₆ alkoxy, —NR^{a}R^{a}, —C(═O)R^{a}, —C(═O)OR^{a}, —NR^{a}C(═O)R^{a}, —C(═O)N(R^{a})₂, —CN, or —NO₂. In some embodiments, R² is substituted or unsubstituted C₁₋₆ alkyl, halogen, hydroxy, substituted or unsubstituted C₁₋₆ alkoxy, —NR^{a}R^{a}, —C(═O)R^{a}, —C(═O)OR^{a}, —NR^{a}C(═O)R^{a}, —C(═O)N(R^{a})₂, —CN, or —NO₂. In some embodiments, R² is substituted or unsubstituted C₁₋₆ alkyl. In some embodiments, R² is unsubstituted C₁_₆ alkyl. In certain embodiments, R² is methyl. In some embodiments, R² is ethyl. In some embodiments, R² is propyl. In some embodiments, R² is isopropyl. In certain embodiments, R² is butyl. In some embodiments, R² is tert-butyl. In some embodiments, R² is substituted C₁₋₆ alkyl. In certain embodiments, R² is substituted methyl (e.g., —CF₃). In some embodiments, R² is substituted ethyl. In some embodiments, R² is substituted propyl. In some embodiments, R² is substituted isopropyl. In certain embodiments, R² is substituted butyl. In some embodiments, R² is substituted or unsubstituted C₁₋₆ alkoxy. In some embodiments, R² is unsubstituted C₁₋₆ alkoxy. In certain embodiments, R² is methoxy. In some embodiments, R² is ethoxy. In some embodiments, R² is propoxy. In certain embodiments, R² is isopropoxy. In some embodiments, R² is butoxy. In certain embodiments, R² is tert-butoxy. In some embodiments, R² is substituted C₁₋₆ alkoxy. In certain embodiments, R² is halogen. In some embodiments, R² is chloro, fluoro, bromo, or iodo. In certain embodiments, R² is chloro. In some embodiments, R² is fluoro. In certain embodiments, R² is bromo. In some embodiments, R² is iodo. In some embodiments, R² is hydroxy. In certain embodiments, R² is —NR^{a}R^{a}. In certain embodiments, R² is —N(CH₃)₂. In certain embodiments, R² is —NH(CH₃). In certain embodiments, R² is —NH₂. In certain embodiments, R² is —C(═O)R^{a}. In certain embodiments, R² is —C(═O)H. In certain embodiments, R² is —C(═O)Me. In certain embodiments, R² is —C(═O)OR^{a}. In certain embodiments, R² is —C(═O)OH. In certain embodiments, R² is —C(═O)OMe. In certain embodiments, R² is —NR^{a}C(═O)R^{a}. In certain embodiments, R² is —NHC(═O)H. In certain embodiments, R² is —NHC(═O)Me. In certain embodiments, R² is —N(Me)C(═O)H. In certain embodiments, R² is —N(Me)C(═O)Me. In certain embodiments, R² is —C(═O)N(R^{a})₂. In certain embodiments, R² is —C(═O)NH₂. In certain embodiments, R² is —C(═O)NHMe. In certain embodiments, R² is —C(═O)N(Me)₂. In certain embodiments, R² is —CN. In some embodiments, R² is —NO₂. In some embodiments, R² is substituted or unsubstituted C₁₋₆ alkyl, halogen, hydroxy, or substituted or unsubstituted C₁₋₆ alkoxy. In certain embodiments, R² is methyl, ethyl, propyl, isopropyl, butyl, tert-butyl, halogen, hydroxy, methoxy, ethoxy, or propoxy. In certain embodiments, R² is methyl, ethyl, propyl, isopropyl, butyl, tert-butyl, fluoro, chloro, bromo, iodo, hydroxy, methoxy, ethoxy, or propoxy. In some embodiments, one instance of R² is substituted or unsubstituted C₁₋₆ alkyl and one instance of R² is hydroxy. In some embodiments, one instance of R² is unsubstituted C₁₋₆ alkyl and one instance of R² is hydroxy. In some embodiments, one instance of R² is methyl and one instance of R² is hydroxy. In some embodiments, one instance of R² is ethyl and one instance of R² is hydroxy.

In some embodiments, the ring bearing R² is of the formula:

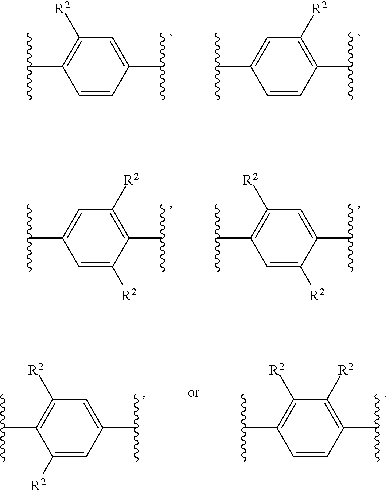

In certain embodiments, the ring bearing R² is of the formula:

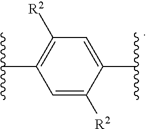

In some embodiments, the ring bearing R² is of the formula:

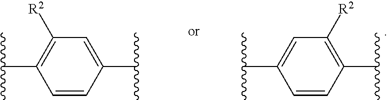

In some embodiments the ring bearing R² is of the formula:

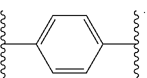 [SMILES: *c1ccc(*)cc1]

In some embodiments, each R³ is independently halogen, hydroxy, —NR^{a}R^{a}, —C(═O)R^{a}, —C(═O)OR^{a}, —CN, or —NO₂. In certain embodiments, R³ is halogen. In some embodiments, R³ is chloro, fluoro, bromo, or iodo. In certain embodiments, R³ is chloro. In some embodiments, R³ is fluoro. In certain embodiments, R³ is bromo. In some embodiments, R³ is iodo. In some embodiments, R³ is hydroxy. In certain embodiments, R³ is —NR^{a}R^{a}. In certain embodiments, R³ is —N(CH₃)₂. In certain embodiments, R³ is —NH(CH₃). In certain embodiments, R³ is —NH₂. In certain embodiments, R³ is —C(═O)R^{a}. In certain embodiments, R³ is —C(═O)H. In certain embodiments, R³ is —C(═O)Me. In certain embodiments, R³ is —C(═O)OR^{a}. In certain embodiments, R³ is —C(═O)OH. In certain embodiments, R³ is —C(═O)OMe. In certain embodiments, R³ is —CN. In some embodiments, R³ is —NO₂. In some embodiments, each instance of R³ is fluoro. In some embodiments, at least one instance of R³ is halogen. In certain embodiments, one instance of R³ is halogen and on instance of R³ is hydroxy. In certain embodiments, one instance of R³ is chloro and on instance of R³ is hydroxy.

In some embodiments, the ring bearing R³ is of the formula:

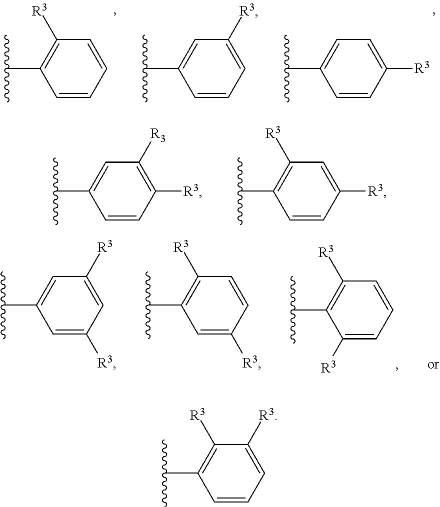

In certain embodiments, the ring bearing R³ is of the formula:

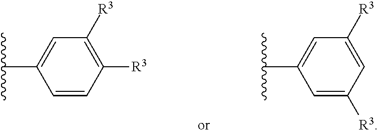

In some embodiments, the ring bearing R³ is of the formula:

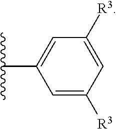

In some embodiments the ring bearing R³ is of the formula:

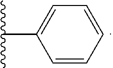 [SMILES: *c1ccccc1]

In some embodiments the ring bearing R³ is of the formula:

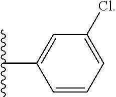 [SMILES: *c1cccc(Cl)c1]

In some embodiments the ring bearing R³ is of the formula:

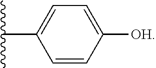 [SMILES: *c1ccc(O)cc1]

In some embodiments the ring bearing R³ is of the formula:

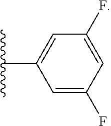 [SMILES: *c1cc(F)cc(F)c1]

In some embodiments the ring bearing R³ is of the formula:

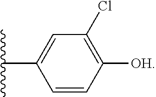 [SMILES: *c1ccc(O)c(Cl)c1]

In some embodiments, each R^{a} is independently H or substituted or unsubstituted C₁₋₆ alkyl. In certain embodiments, R^{a} is H. In some embodiments, R^{a} is substituted or unsubstituted C₁₋₆ alkyl. In some embodiments, R^{a} is unsubstituted C₁₋₆ alkyl. In some embodiments, R^{a} is substituted C₁₋₆ alkyl. In some embodiments, R^{a} is methyl. In certain embodiments, R^{a} is ethyl. In certain embodiments, each instance of R^{a} is H. In some embodiments, each instance of R^{a} is substituted or unsubstituted C₁₋₆ alkyl. In some embodiments, each instance of R^{a} is unsubstituted C₁₋₆ alkyl. In some embodiments, each instance of R^{a} is substituted C₁₋₆ alkyl. In some embodiments, each instance of R^{a} is methyl. In certain embodiments, each instance of R^{a} is ethyl.

In some embodiments, n is 0, 1, 2, 3, or 4. In certain embodiments, n is 0, 1, or 2. In some embodiments, n is 1. In certain embodiments, n is 2.

In some embodiments, m is 0, 1, 2, 3, or 4. In certain embodiments, m is 0, 1, or 2. In some embodiments, m is 1. In some embodiments, m is 2.

In some embodiments, p is 0, 1, 2, 3, 4, or 5. In certain embodiments, p is 0, 1, 2, or 3.

In some embodiments, p is 0, 1, or 2. In certain embodiments, p is 1 or 2.

In certain embodiments, Formula (I) is of the Formula (I-a):

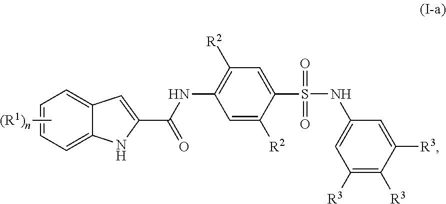

or pharmaceutically acceptable salts, solvates, hydrates, polymorphs, co-crystals, tautomers, isotopologues, stereoisomers, or prodrugs thereof.

In certain embodiments, Formula (I) is of the Formula (I-b):

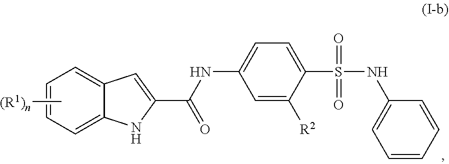

or pharmaceutically acceptable salts, solvates, hydrates, polymorphs, co-crystals, tautomers, isotopologues, stereoisomers, or prodrugs thereof.

In certain embodiments, Formula (I) is of the Formula (I-c):

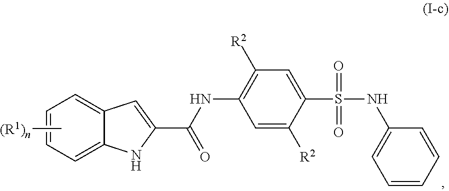

or pharmaceutically acceptable salts, solvates, hydrates, polymorphs, co-crystals, tautomers, isotopologues, stereoisomers, or prodrugs thereof.

In certain embodiments, Formula (I) is of the Formula (I-d):

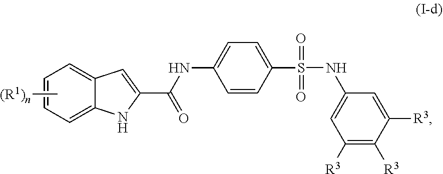

or pharmaceutically acceptable salts, solvates, hydrates, polymorphs, co-crystals, tautomers, isotopologues, stereoisomers, or prodrugs thereof.

In certain embodiments, Formula (I) is of the Formula (I-e):

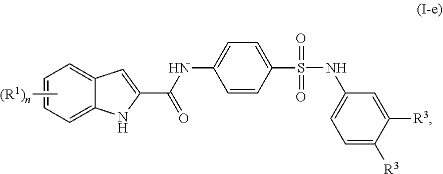

or pharmaceutically acceptable salts, solvates, hydrates, polymorphs, co-crystals, tautomers, isotopologues, stereoisomers, or prodrugs thereof.

In certain embodiments, Formula (I) is of the Formula (I-f):

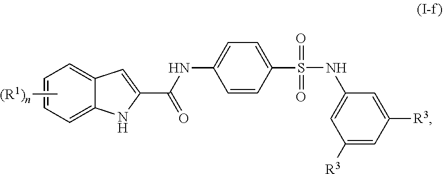

or pharmaceutically acceptable salts, solvates, hydrates, polymorphs, co-crystals, tautomers, isotopologues, stereoisomers, or prodrugs thereof.

In certain embodiments, Formula (I) is of the Formula (I-g):

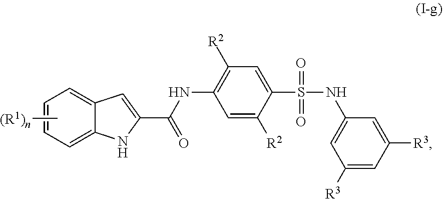

or pharmaceutically acceptable salts, solvates, hydrates, polymorphs, co-crystals, tautomers, isotopologues, stereoisomers, or prodrugs thereof.

In certain embodiments, Formula (I) is of the Formula (I-h):

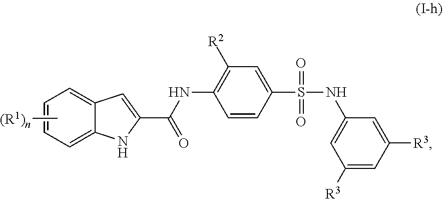

or pharmaceutically acceptable salts, solvates, hydrates, polymorphs, co-crystals, tautomers, isotopologues, stereoisomers, or prodrugs thereof.

In certain embodiments, Formula (I) is of the Formula (I-i):

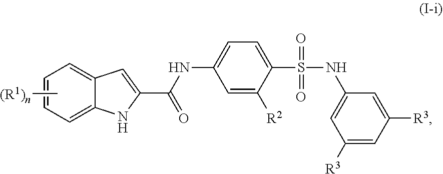

or pharmaceutically acceptable salts, solvates, hydrates, polymorphs, co-crystals, tautomers, isotopologues, stereoisomers, or prodrugs thereof.

In certain embodiments, a compound of Formula (0) is of the formula:

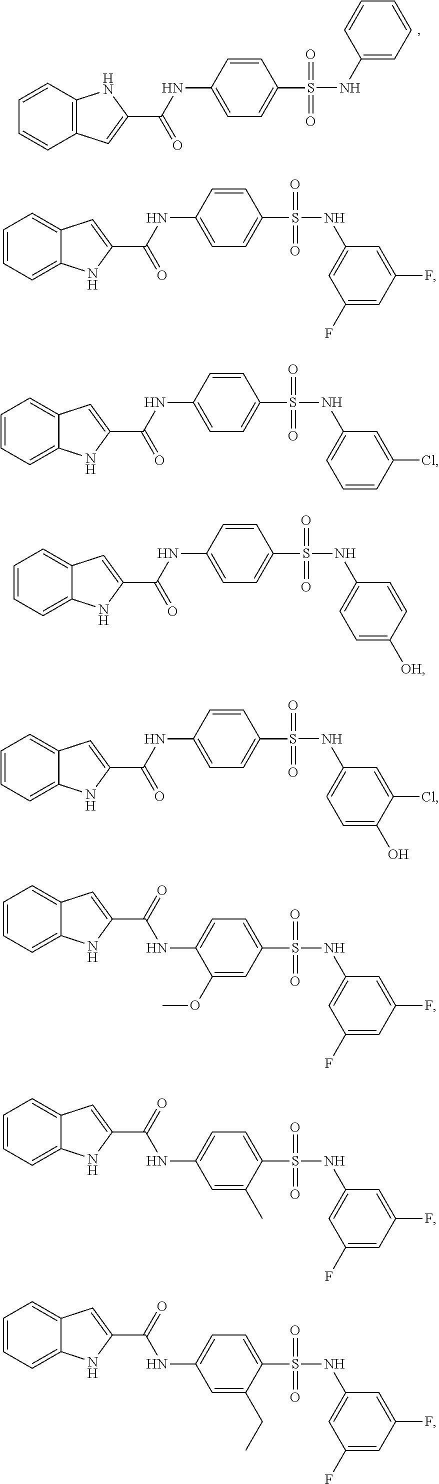

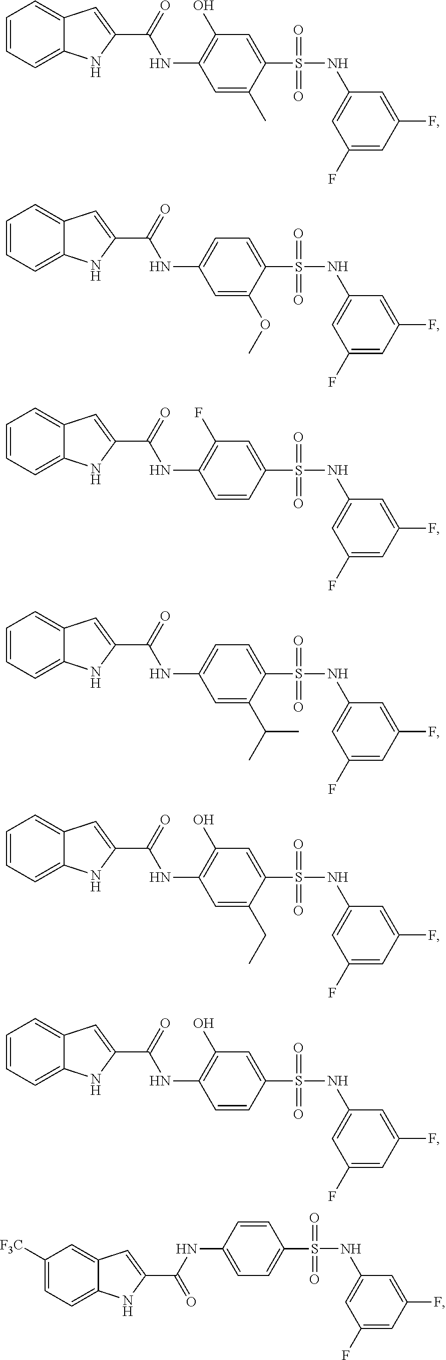

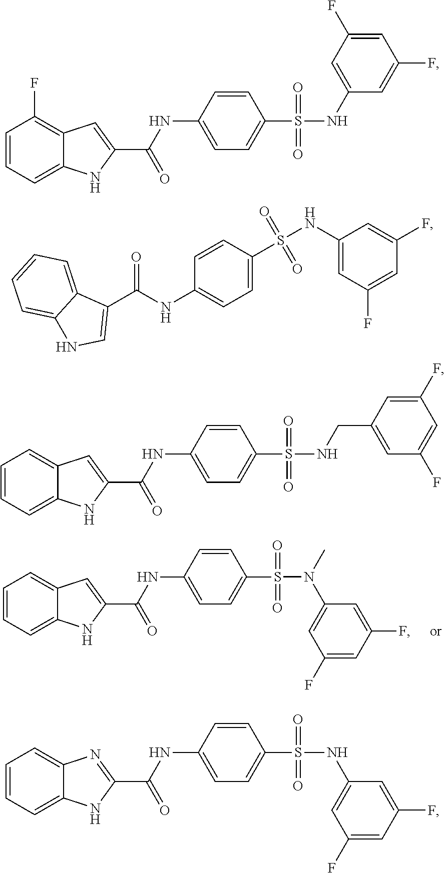

or a salt thereof. In some embodiments, a compound of Formula (0) is of the formula:

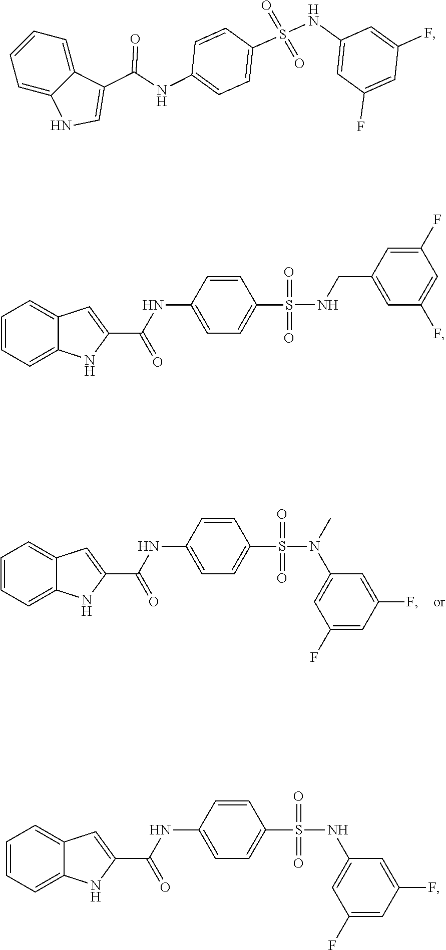 [SMILES: O=C(Nc1ccc(S(=O)(=O)Nc2cc(F)cc(F)c2)cc1)c1c[nH]c2ccccc12 ; O=C(Nc1ccc(S(=O)(=O)NCc2cc(F)cc(F)c2)cc1)c1cc2ccccc2[nH]1 ; CN(c1cc(F)cc(F)c1)S(=O)(=O)c1ccc(NC(=O)c2cc3ccccc3[nH]2)cc1 ; O=C(Nc1ccc(S(=O)(=O)Nc2cc(F)cc(F)c2)cc1)c1cc2ccccc2[nH]1]

or a salt thereof.

In certain embodiments, a compound of Formula (I) is of the formula:

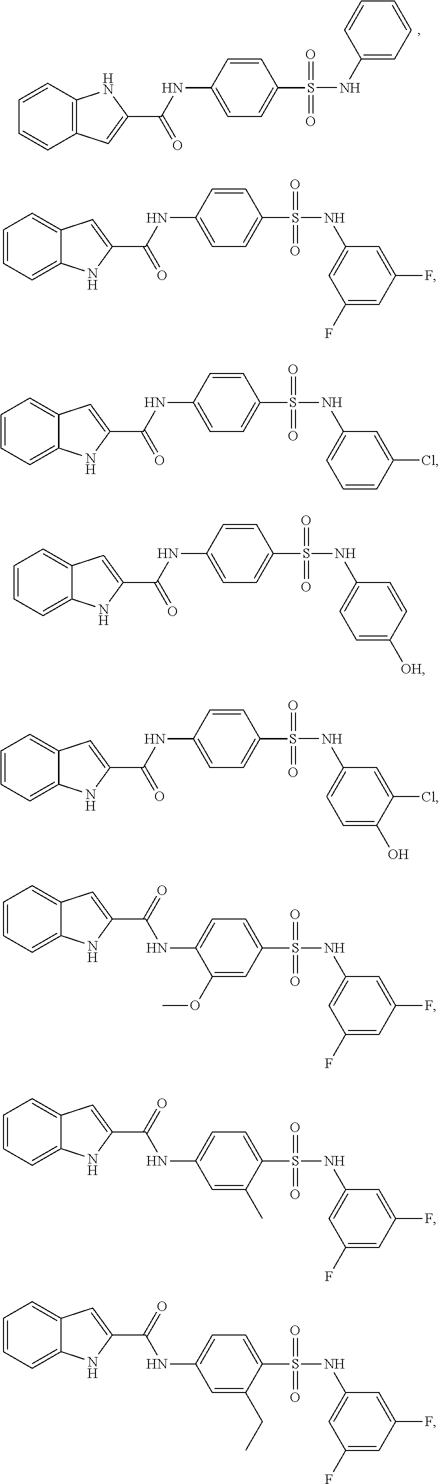

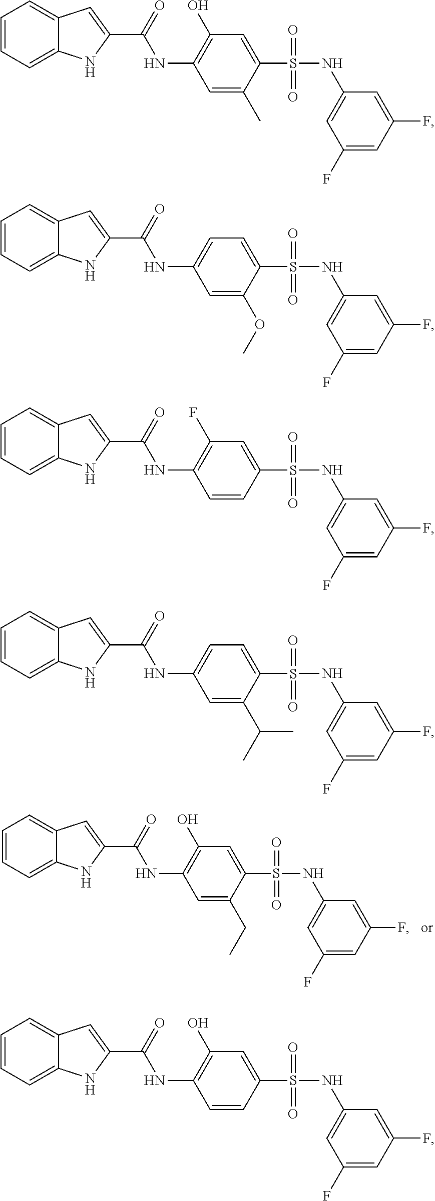 [SMILES: Cc1cc(NC(=O)c2cc3ccccc3[nH]2)c(O)cc1S(=O)(=O)Nc1cc(F)cc(F)c1 ; COc1cc(NC(=O)c2cc3ccccc3[nH]2)ccc1S(=O)(=O)Nc1cc(F)cc(F)c1 ; O=C(Nc1ccc(S(=O)(=O)Nc2cc(F)cc(F)c2)cc1F)c1cc2ccccc2[nH]1 ; CC(C)c1cc(NC(=O)c2cc3ccccc3[nH]2)ccc1S(=O)(=O)Nc1cc(F)cc(F)c1 ; CCc1cc(NC(=O)c2cc3ccccc3[nH]2)c(O)cc1S(=O)(=O)Nc1cc(F)cc(F)c1 ; O=C(Nc1ccc(S(=O)(=O)Nc2cc(F)cc(F)c2)cc1O)c1cc2ccccc2[nH]1]

or a salt thereof. In certain embodiments, a compound of Formula (I) is of the formula:

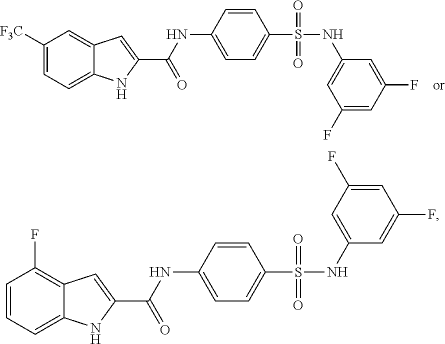

or a salt thereof. In certain embodiments, a compound of Formula (I) is of the formula:

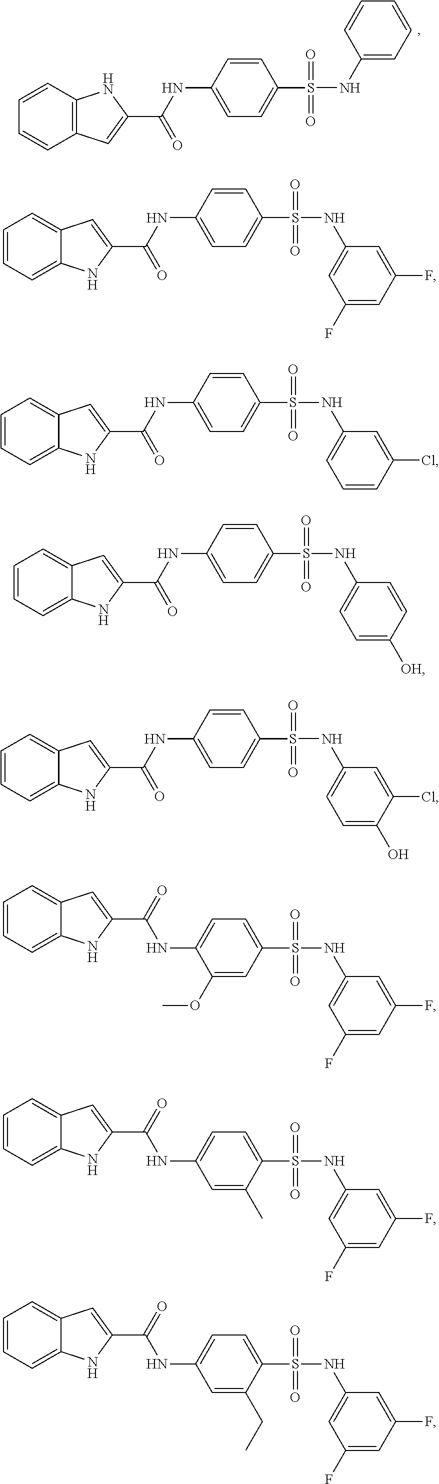

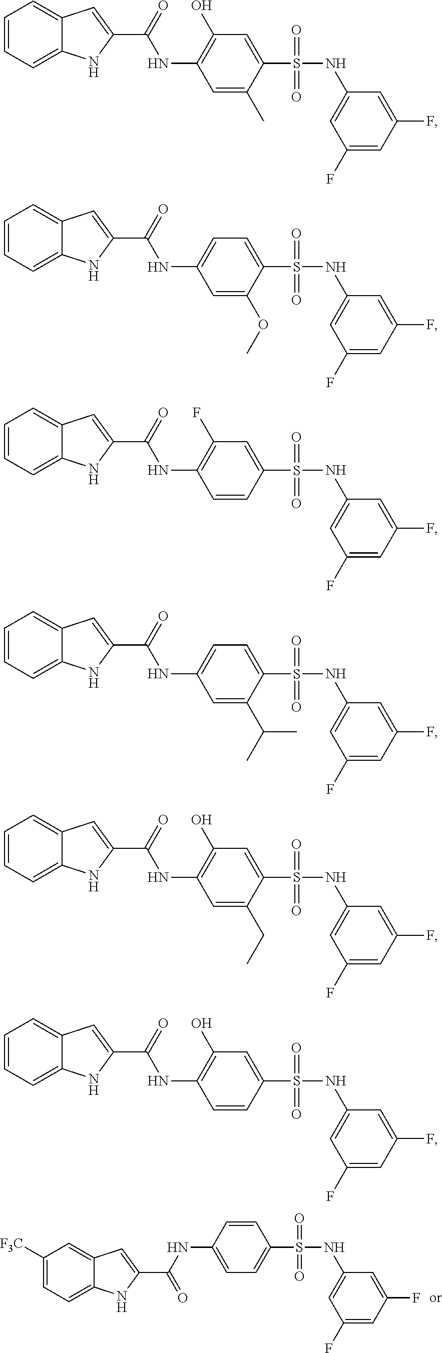

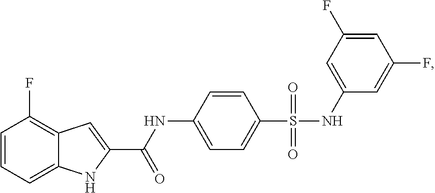 [SMILES: O=C(Nc1ccc(S(=O)(=O)Nc2cc(F)cc(F)c2)cc1)c1cc2c(F)cccc2[nH]1]

In certain embodiments, a compound described herein is a compound of Formula (0), or a pharmaceutically acceptable salt, solvate, hydrate, polymorph, co-crystal, tautomer, stereoisomer, isotopologue, or prodrug thereof. In certain embodiments, a compound of Formula (0), or a pharmaceutically acceptable salt thereof.

In certain embodiments, a compound described herein is a compound of Formula (I), or a pharmaceutically acceptable salt, solvate, hydrate, polymorph, co-crystal, tautomer, stereoisomer, isotopologue, or prodrug thereof. In certain embodiments, a compound of Formula (I), or a pharmaceutically acceptable salt thereof.

In certain embodiments, a compound described herein is any of the compounds I-1 to I-14, found in Table 1, or a pharmaceutically acceptable salt, solvate, hydrate, polymorph, co-crystal, tautomer, stereoisomer, isotopologue, or prodrug thereof. In certain embodiments, a compound described herein is any of the compounds I-1 to I-14, or a pharmaceutically acceptable salt thereof.

TABLE 1
Compounds I-1 to I-14 of the disclosure

| Compound | Compound | |
|---|---|---|
| Number | Name | Structure: |
| I-1 | RJB-2-88 | 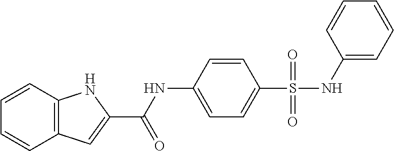 [SMILES: O=C(Nc1ccc(S(=O)(=O)Nc2ccccc2)cc1)c1cc2ccccc2[nH]1] |
| I-2 | RJB-2-145 and RJB-145 | 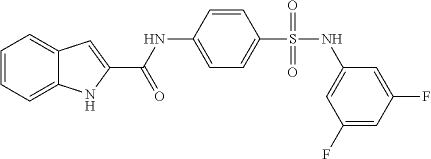 [SMILES: O=C(Nc1ccc(S(=O)(=O)Nc2cc(F)cc(F)c2)cc1)c1cc2ccccc2[nH]1] |
| I-3 | RJB-2-146 | 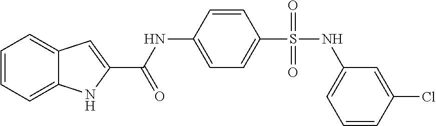 [SMILES: O=C(Nc1ccc(S(=O)(=O)Nc2cccc(Cl)c2)cc1)c1cc2ccccc2[nH]1] |
| I-4 | RJB-2-151 | 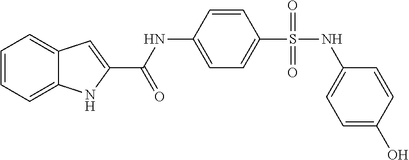 [SMILES: O=C(Nc1ccc(S(=O)(=O)Nc2ccc(O)cc2)cc1)c1cc2ccccc2[nH]1] |
| I-5 | RJB-2-155 | 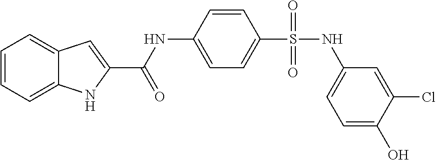 [SMILES: O=C(Nc1ccc(S(=O)(=O)Nc2ccc(O)c(Cl)c2)cc1)c1cc2ccccc2[nH]1] |
| I-6 | RJB-3-77 | 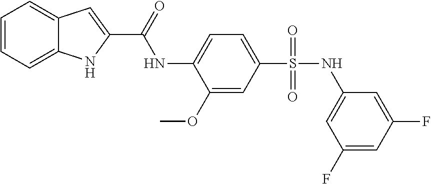 [SMILES: COc1cc(S(=O)(=O)Nc2cc(F)cc(F)c2)ccc1NC(=O)c1cc2ccccc2[nH]1] |
| I-7 | RJB-3-161 | 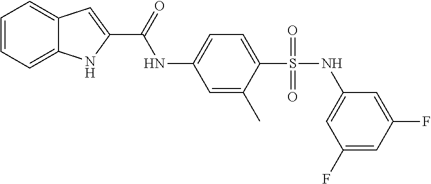 [SMILES: Cc1cc(NC(=O)c2cc3ccccc3[nH]2)ccc1S(=O)(=O)Nc1cc(F)cc(F)c1] |
| I-8 | RJB-3-164 | 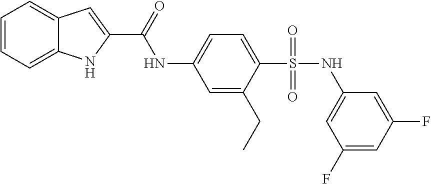 [SMILES: CCc1cc(NC(=O)c2cc3ccccc3[nH]2)ccc1S(=O)(=O)Nc1cc(F)cc(F)c1] |
| I-9 | RJB-3-169 | 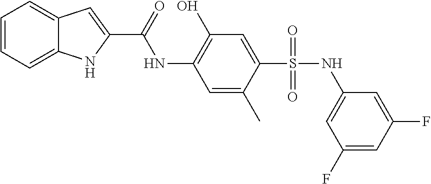 [SMILES: Cc1cc(NC(=O)c2cc3ccccc3[nH]2)c(O)cc1S(=O)(=O)Nc1cc(F)cc(F)c1] |
| I-10 | RJB-3-178 | 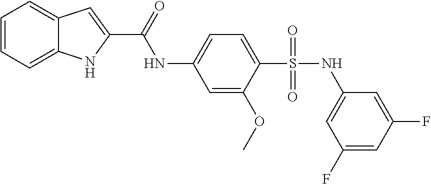 [SMILES: COc1cc(NC(=O)c2cc3ccccc3[nH]2)ccc1S(=O)(=O)Nc1cc(F)cc(F)c1] |
| I-11 | RJB-3-180 | 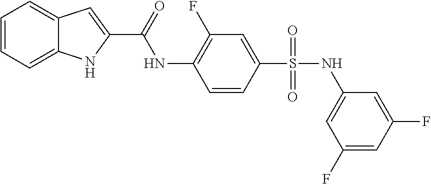 [SMILES: O=C(Nc1ccc(S(=O)(=O)Nc2cc(F)cc(F)c2)cc1F)c1cc2ccccc2[nH]1] |
| I-12 | RJB-3-184 | 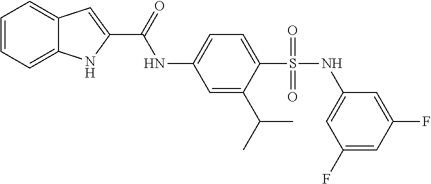 [SMILES: CC(C)c1cc(NC(=O)c2cc3ccccc3[nH]2)ccc1S(=O)(=O)Nc1cc(F)cc(F)c1] |
| I-13 | RJB-3-201 | 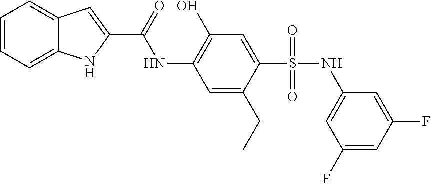 [SMILES: CCc1cc(NC(=O)c2cc3ccccc3[nH]2)c(O)cc1S(=O)(=O)Nc1cc(F)cc(F)c1] |
| I-14 | RJB-3-214 | 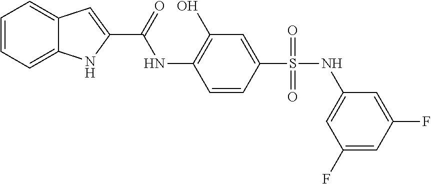 [SMILES: O=C(Nc1ccc(S(=O)(=O)Nc2cc(F)cc(F)c2)cc1O)c1cc2ccccc2[nH]1] |

In certain embodiments, a compound disclosed herein is of Formula (I) (e.g., compounds I-1 to I-14), or a pharmaceutically acceptable salt, solvate, hydrate, polymorph, co-crystal, tautomer, stereoisomer, isotopologue, or prodrug thereof. In certain embodiments, a compound disclosed herein is of Formula (I) (e.g., compounds I-1 to I-14), or a pharmaceutically acceptable salt, solvate, hydrate, co-crystal, tautomer, stereoisomer, or prodrug thereof. In certain embodiments, a compound disclosed herein is of Formula (I) (e.g., compounds I-1 to I-14), or a pharmaceutically acceptable salt, solvate, hydrate, co-crystal, tautomer, or stereoisomer thereof. In certain embodiments, a compound disclosed herein is of Formula (I) (e.g., compounds I-1 to I-14), or a pharmaceutically acceptable salt, tautomer, or stereoisomer thereof. In certain embodiments, a compound disclosed herein is of Formula (I) (e.g., compounds I-1 to I-14), or a pharmaceutically acceptable salt, solvate, hydrate, or co-crystal thereof. In certain embodiments, a compound disclosed herein is of Formula (I) (e.g., compounds I-1 to I-14), or a pharmaceutically acceptable salt, solvate, or hydrate thereof.

### Methods of Treatment and Uses

The compounds described herein may be useful in treating and/or preventing diseases or disorders including, but not limited to, addiction (e.g. addiction to a substance (e.g. addiction to cocaine, amphetamines, methamphetamine, methylphenidate, nicotine, alcohol, prescription medication, marijuana, tobacco, food, or an opioid selected from heroin, fentanyl, codeine, hydrocodone, morphine, oxycodone, hydromorphone, and methadone)), stress-related disorders (e.g. chronic stress, PTSD, panic disorder, anxiety, or general anxiety disorders), eating disorders (e.g. orthorexia, or anorexia nervosa), or pain-related diseases or disorders (e.g. chronic pain, arthritis (e.g., rheumatoid arthritis), inflammatory pain, musculoskeletal pain, primary headache disorders, or migraines), or diseases and disorders associated with the activity of a PACAP receptor in a subject, or modulating the activity of a PACAP receptor in a subject, biological sample, tissue, or cell.

In one aspect, the disclosure provides a method of treating a subject suffering from or susceptible to a disease or disorder. In one aspect, the disclosure provides a method of treating a subject suffering from or susceptible to a neurological disorder (e.g. stress-related disorders (e.g. chronic stress, post-traumatic stress disorder (PTSD), panic disorder, anxiety, or general anxiety disorder), eating disorders (e.g. orthorexia, or anorexia nervosa), pain-related disorders (e.g. chronic pain, arthritis (e.g., rheumatoid arthritis), inflammatory pain, musculoskeletal pain, primary headache disorders, or migraines), panic disorders, panic attacks, or addiction (e.g. addiction to a substance (e.g. addiction to cocaine, amphetamines, methamphetamine, methylphenidate, nicotine, alcohol, prescription medication, marijuana, tobacco, food, or an opioid selected from heroin, fentanyl, codeine, hydrocodone, morphine, oxycodone, hydromorphone, and methadone)), the method comprising administering to the subject in need thereof a therapeutically effective amount of a compound of Formula (I), or a pharmaceutically acceptable salt, solvate, hydrate, polymorph, co-crystal, tautomer, stereoisomer, isotopologue, or prodrug thereof. In certain aspects, the method treats addiction by preventing relapse of an addiction. In another aspect, the method treats addiction by lowering the incidence of relapse, as compared to a control population not receiving treatment via the method. In certain aspects, the method treats addiction by preventing stress-induced relapse of an addiction. In some embodiments, the disease or disorder is chronic stress. In some embodiments, the disease or disorder is post-traumatic stress disorder (PTSD). In some embodiments, the disease or disorder is panic disorder. In some embodiments, the disease or disorder is anxiety. In some embodiments, the disease or disorder is general anxiety disorder. In some embodiments, the disease or disorder is chronic stress. orthorexia. In some embodiments, the disease or disorder is anorexia nervosa. In some embodiments, the disease or disorder is chronic stress. chronic pain. In some embodiments, the disease or disorder is arthritis. In some embodiments, the disease or disorder is rheumatoid arthritis. In some embodiments, the disease or disorder is inflammatory pain. In some embodiments, the disease or disorder is musculoskeletal pain. In some embodiments, the disease or disorder is primary headache disorder. In some embodiments, the disease or disorder is migraine. In some embodiments, the disease or disorder is panic attacks. In some embodiments, the disease or disorder is addiction to cocaine. In some embodiments, the disease or disorder is addiction to amphetamines. In some embodiments, the disease or disorder is addiction to methamphetamine. In some embodiments, the disease or disorder is addiction to methylphenidate. In some embodiments, the disease or disorder is addiction to nicotine. In some embodiments, the disease or disorder is addiction to alcohol. In some embodiments, the disease or disorder is addiction to prescription medication. In some embodiments, the disease or disorder is addiction to marijuana. In some embodiments, the disease or disorder is addiction to tobacco. In some embodiments, the disease or disorder is addiction to food. In some embodiments, the disease or disorder is addiction to an opioid selected from heroin, fentanyl, codeine, hydrocodone, morphine, oxycodone, hydromorphone, and methadone.

In one aspect, the method comprises administering to a subject a therapeutically effective amount of compound I-1, or a salt thereof. In one aspect, the method comprises administering to a subject a therapeutically effective amount of compound I-2, or a salt thereof. In one aspect, the method comprises administering to a subject a therapeutically effective amount of compound I-3, or a salt thereof. In one aspect, the method comprises administering to a subject a therapeutically effective amount of compound I-4, or a salt thereof. In one aspect, the method comprises administering to a subject a therapeutically effective amount of compound I-5, or a salt thereof. In one aspect, the method comprises administering to a subject a therapeutically effective amount of compound I-6, or a salt thereof. In one aspect, the method comprises administering to a subject a therapeutically effective amount of compound I-7, or a salt thereof. In one aspect, the method comprises administering to a subject a therapeutically effective amount of compound I-8, or a salt thereof. In one aspect, the method comprises administering to a subject a therapeutically effective amount of compound I-9, or a salt thereof. In one aspect, the method comprises administering to a subject a therapeutically effective amount of compound I-10, or a salt thereof. In one aspect, the method comprises administering to a subject a therapeutically effective amount of compound I-11, or a salt thereof. In one aspect, the method comprises administering to a subject a therapeutically effective amount of compound I-12, or a salt thereof. In one aspect, the method comprises administering to a subject a therapeutically effective amount of compound I-13, or a salt thereof. In one aspect, the method comprises administering to a subject a therapeutically effective amount of compound I-14, or a salt thereof.

In another aspect, the disclosure provides a method of modulating the activity of a PACAP-receptor (e.g. PAC1) comprising administering an effective amount of any one of the compounds of Formula (I), or a salt thereof. In another aspect, the modulation is inhibition. In another aspect, the modulation occurs in a biological sample (e.g. tissue or cell). In another aspect, the modulation occurs in a subject. In another aspect, PAC1 receptor antagonism can prevent relapse of an addiction. In certain aspects, PAC1 receptor antagonism can prevent stress-induced relapse of an addiction.

The disclosure provides a method of treating a subject suffering from or susceptible to a PACAP receptor-mediated disease or disorder, the method comprising administering to the subject in need thereof a therapeutically effective amount of any one of compounds of Formula (I), or a salt thereof. In another aspect, the PACAP receptor-mediated disorder is a neurological disorder. In another aspect, the PACAP receptor-mediated disease or disorder is a stress-related disorder (e.g. chronic stress, post-traumatic stress disorder (PTSD), panic disorder, anxiety, or general anxiety disorder). In another aspect, the PACAP receptor-mediated disease or disorder is a pain-related disorders (e.g. chronic pain, arthritis (e.g., rheumatoid arthritis), inflammatory pain, musculoskeletal pain, primary headache disorders, or migraines).

Also provided herein are methods for modulating a pituitary adenylate cyclase activating polypeptide (PACAP) receptor, comprising contacting a PAC1 receptor with a compound disclosed herein, or pharmaceutically acceptable salt thereof, or a pharmaceutical composition as provided herein. In some embodiments, the modulating is inhibiting. Thus, in some embodiments are methods for inhibiting a pituitary adenylate cyclase activating polypeptide (PACAP) receptor, comprising contacting a PAC1 receptor with a compound disclosed herein, or pharmaceutically acceptable salt thereof, or a pharmaceutical composition as provided herein.

The disclosure further provides methods for modulating a PAC1 receptor, comprising contacting a PAC1 receptor with a compound disclosed herein, or pharmaceutically acceptable salt thereof, or a pharmaceutical composition as provided herein. In some embodiments, the modulating is inhibiting. Thus, in some embodiments, provided herein are methods for inhibiting a PAC1 receptor, comprising contacting a PAC1 receptor with a compound disclosed herein, or pharmaceutically acceptable salt thereof, or a pharmaceutical composition as provided herein.

The disclosure further provides, methods of treating a disease or disorder in a subject by modulating a pituitary adenylate cyclase activating polypeptide (PACAP) receptor, the method comprising administering to the subject a therapeutically effective amount of a compound disclosed herein, or pharmaceutically acceptable salt thereof, or a pharmaceutical composition as provided herein. In some embodiments, the modulating is inhibiting. Thus, in some embodiments, provided herein are methods of treating a disease or disorder in a subject by inhibiting a pituitary adenylate cyclase activating polypeptide (PACAP) receptor, the method comprising administering to the subject a therapeutically effective amount of a compound disclosed herein, or pharmaceutically acceptable salt thereof, or a pharmaceutical composition as provided herein.

Also provided are methods of treating a disease or disorder in a subject by modulating a PAC1 receptor, the method comprising administering to the subject a therapeutically effective amount of a compound disclosed herein, or pharmaceutically acceptable salt thereof, or a pharmaceutical composition as provided herein. In some embodiments, the modulating is inhibiting. Thus, in some embodiments, provided herein are methods of treating a disease or disorder in a subject by inhibiting a PAC1 receptor, the method comprising administering to the subject a therapeutically effective amount of a compound disclosed herein, or pharmaceutically acceptable salt thereof, or a pharmaceutical composition as provided herein.

In another aspect, the method comprises administering to a subject a therapeutically effective amount of a combination of any one of the compounds of Formula (I), or salts thereof. In another aspect, the neurological disorder is a stress-related disorder (e.g. chronic stress, post-traumatic stress disorder (PTSD), panic disorder, anxiety, or general anxiety disorder). In another aspect, the neurological disorder is an eating disorder (e.g. orthorexia, or anorexia nervosa). In another aspect, the neurological disorder is a pain-related disorder (e.g. chronic pain, arthritis (e.g., rheumatoid arthritis), inflammatory pain, musculoskeletal pain, primary headache disorder, or migraine). In another aspect, the neurological disorder is a panic disorder (e.g., panic attacks). In another aspect, the neurological disorder is addiction (e.g. addiction to a substance (e.g. addiction to cocaine, amphetamines, methamphetamine, methylphenidate, nicotine, alcohol, prescription medication, marijuana, tobacco, food, or an opioid selected from heroin, fentanyl, codeine, hydrocodone, morphine, oxycodone, hydromorphone, and methadone)).

In certain embodiments, a compound of Formula (I), is administered in combination with one or more additional pharmaceutical agents. The additional pharmaceutical agent may also be a PACAP receptor antagonist. In certain embodiments, the additional pharmaceutical agent is an additional compound of Formula (I), or a salt thereof. In certain embodiments, the additional pharmaceutical agent is an inhibitor of a kinase. In certain embodiments, the additional pharmaceutical agent is an inhibitor of MEK/ERK. In certain embodiments, the additional pharmaceutical agent is an anti-inflammatory agent, pain-relieving agent, antihistamine, beta-blocker, anxiolytic, anti-depressant, selective serotonin reuptake inhibitor (SSRI), serotonin and norepinephrine reuptake inhibitor (SNRI), sympatholytic, monoamine oxidase inhibitor (MAOI), opioid, carbamate, benzodiazepine, barbiturate, GABAergic drug, antipsychotic, or other agent.

The additional pharmaceutical agents include, but are not limited to, anti-anxiety agents, anti-inflammatory agents, antihistamines, anxiolytics, antidepressants, anti-allergic agents, pain-relieving agents, and any combination thereof. In certain embodiments, a pharmaceutical composition described herein further comprises a combination of the additional pharmaceutical agents described herein.

The inventive compounds or compositions may synergistically modulate a PACAP receptor induced by the additional pharmaceutical agent(s) in the biological sample or subject. Thus, the combination of the inventive compounds or compositions and the additional pharmaceutical agent(s) may be useful in treating neurological diseases resistant to a treatment using the additional pharmaceutical agent(s) without the inventive compounds or compositions.

In another aspect, the disclosure provides a kit for treating a neurological disease or disorder comprising a therapeutically effective amount of a compound of Formula (I), or a salt thereof, and instructions for administration of the compound, or a salt thereof, to a subject. In another aspect, the neurological disease or disorder is a stress-related disorder (e.g. chronic stress, post-traumatic stress disorder (PTSD), panic disorder, anxiety, or general anxiety disorder), eating disorders (e.g. orthorexia, or anorexia nervosa), pain-related disorder (e.g. chronic pain, arthritis (e.g., rheumatoid arthritis), inflammatory pain, musculoskeletal pain, primary headache disorders, or migraines), panic disorders, panic attacks, or addiction (e.g. addiction to a substance (e.g. addiction to cocaine, amphetamines, methamphetamine, methylphenidate, nicotine, alcohol, prescription medication, marijuana, tobacco, food, or an opioid selected from heroin, fentanyl, codeine, hydrocodone, morphine, oxycodone, hydromorphone, and methadone)).

In another aspect, the disclosure provides a method of ameliorating a disorder or symptom thereof in a subject, the method comprising administering to the subject in need thereof a therapeutically effective amount of a compound of Formula (I), or a salt thereof. In another aspect, the disease is a neurological disease or disorder.

Determination of a therapeutically effective amount or a prophylactically effective amount of the compound of the disclosure, can be readily made by the physician or veterinarian (the “attending clinician”), as one skilled in the art, by the use of known techniques and by observing results obtained under analogous circumstances. The dosages may be varied depending upon the requirements of the patient in the judgment of the attending clinician; the severity of the condition being treated and the particular compound being employed. In determining the therapeutically effective amount or dose, and the prophylactically effective amount or dose, a number of factors are considered by the attending clinician, including, but not limited to: the specific neurological disorder involved; pharmacodynamics characteristics of the particular agent and its mode and route of administration; the desired time course of treatment; the species of mammal; its size, age, and general health; the specific disease involved; the degree of or involvement or the severity of the disease; the response of the individual patient; the particular compound administered; the mode of administration; the bioavailability characteristics of the preparation administered; the dose regimen selected; the kind of concurrent treatment (i.e., the interaction of the compound of the disclosure with other co-administered therapeutics); and other relevant circumstances.

Treatment can be initiated with smaller dosages, which are less than the optimum dose of the compound. Thereafter, the dosage may be increased by small increments until the optimum effect under the circumstances is reached. For convenience, the total daily dosage may be divided and administered in portions during the day if desired. A therapeutically effective amount and a prophylactically effective amount of a compound of the disclosure is expected to vary from about 0.1 milligram per kilogram of body weight per day (mg/kg/day) to about 100 mg/kg/day.

Compounds determined to be effective for the prevention or treatment of neurological disorders in animals, e.g., dogs, chickens, and rodents, may also be useful in treatment of neurological disorders in humans. Those skilled in the art of treating neurological disorders in humans will know, based upon the data obtained in animal studies, the dosage and route of administration of the compound to humans. In general, the dosage and route of administration in humans is expected to be similar to that in animals.

The identification of those patients who are in need of prophylactic treatment for neurological disorders is well within the ability and knowledge of one skilled in the art. Certain of the methods for identification of patients which are at risk of developing neurological disorders which can be treated by the subject method are appreciated in the medical arts, such as family history, and the presence of risk factors associated with the development of that disease state in the subject patient. A clinician skilled in the art can readily identify such candidate patients, by the use of, for example, clinical tests, physical examination and medical/family history.

A method of assessing the efficacy of a treatment in a subject includes determining the pre-treatment extent of a neurological disorder by methods well known in the art and then administering a therapeutically effective amount of a compound of Formula (I), according to the disclosure to the subject. After an appropriate period of time after the administration of the compound (e.g., 1 day, 1 week, 2 weeks, one month, six months), the extent of the neurological disorder is determined again. The modulation (e.g., decrease) of the extent of the neurological disorder indicates efficacy of the treatment. The extent of the neurological disorder may be determined periodically throughout treatment. For example, the extent of the neurological disorder may be checked every few hours, days or weeks to assess the further efficacy of the treatment. A decrease in extent of the neurological disorder indicates that the treatment is efficacious. The method described may be used to screen or select patients that may benefit from treatment with a PACAP receptor antagonist for a neurological disorder.

As used herein, “obtaining a biological sample from a subject,” includes obtaining a sample for use in the methods described herein. A biological sample is described above.

In another aspect, a compound of the disclosure or pharmaceutical composition thereof is packaged in a therapeutically effective amount with a pharmaceutically acceptable carrier or diluent. The composition may be formulated for treating a subject suffering from or susceptible to a neurological disorder, and packaged with instructions to treat a subject suffering from or susceptible to a neurological disorder.

In another aspect, methods of treating a neurological disorder in a subject include administering an effective amount of a compound of the disclosure to the subject. The administration may be by any route of administering known in the pharmaceutical arts. The subject may have a neurological disorder, may be at risk of developing a neurological disorder, or may need prophylactic treatment prior to anticipated or unanticipated exposure to conditions capable of increasing susceptibility to neurological disorder.

In one aspect, a method of monitoring the progress of a subject being treated includes determining the pre-treatment status of the neurological disorder, administering a therapeutically effective amount of compound of the disclosure to the subject, and determining the status of the neurological disorder after an initial period of treatment, wherein the modulation of the status indicates efficacy of the treatment.

The subject may be at risk of a neurological disorder, may be exhibiting symptoms of a neurological disorder, may be susceptible to a neurological disorder and/or may have been diagnosed with a neurological disorder (e.g. stress-related disorders (e.g. chronic stress, post-traumatic stress disorder (PTSD), panic disorder, anxiety, or general anxiety disorder), eating disorders (e.g. orthorexia, or anorexia nervosa), pain-related disorders (e.g. chronic pain, arthritis (e.g., rheumatoid arthritis), inflammatory pain, musculoskeletal pain, primary headache disorders, or migraines), panic disorders, panic attacks, or addiction (e.g. addiction to a substance (e.g. addiction to cocaine, amphetamines, methamphetamine, methylphenidate, nicotine, alcohol, prescription medication, marijuana, tobacco, food, or an opioid selected from heroin, fentanyl, codeine, hydrocodone, morphine, oxycodone, hydromorphone, and methadone)).

The initial period of treatment may be the time in which it takes to establish a stable and/or therapeutically effective blood serum level of any of the compounds, compound combinations, dosing regimens, or pharmaceutical compositions delineated herein, or the time in which it take for the subject to clear a substantial portion of the compound, or any period of time selected by the subject or healthcare professional that is relevant to the treatment.

If the modulation of the status indicates that the subject may have a favorable clinical response to the treatment, the subject may be treated with the compound. For example, the subject can be administered a therapeutically effective dose or doses of the compound.

In another aspect, the disclosure provides antagonists for PACAP-receptors (e.g. PAC1) and methods for their use in a biological sample (e.g. tissue or cell). The methods include contacting the biological sample (e.g. tissue or cell) with an effective amount of any of the compounds of Formula (I), compound combinations, dosing regimens, or pharmaceutical compositions delineated herein, such that the signaling of the PACAP-receptor is reduced. The contacting may be in vitro, e.g., by addition of the compound to a fluid surrounding the cells, for example, to the growth media in which the cells are living or existing. The contacting may also be by directly contacting the compound to the cells. Alternately, the contacting may be in vivo, e.g., by passage of the compound through a subject; for example, after administration, depending on the route of administration, the compound may travel through the digestive tract or the blood stream or may be applied or administered directly to cells in need of treatment.

The PACAP-receptor may be within a cell, isolated from a cell, recombinantly expressed, purified or isolated from a cell or recombinant expression system or partially purified or isolated from a cell or recombinant expression system.

The contacting may be in vitro, e.g., by addition of the compound to a solution containing purified PACAP-receptor, or, if the PACAP-receptor is present in cells, by adding the compound to a fluid surrounding the cells, for example, to the growth media in which the cells are living or existing. The contacting may also be by directly contacting the compound to the cells. Alternately, the contacting may be in vivo, e.g., by passage of the compound through a subject; for example, after administration, depending on the route of administration, the compound may travel through the digestive tract or the blood stream or may be applied or administered directly to cells in need of treatment.

Kits of the disclosure include kits for treating a neurological disorder in a subject. The disclosure also includes kits for regulation of PACAP-receptor function, assessing the efficacy of a treatment for a neurological disorder in a subject, monitoring the progress of a subject being treated for a neurological disorder, selecting a subject with a neurological disorder for treatment according to the disclosure, and/or treating a subject suffering from or susceptible to a neurological disorder. The kit may include any of the compounds, compound combinations, dosing regimens, or pharmaceutical compositions delineated herein and instructions for use. The instructions for use may include information on dosage, method of delivery, storage of the kit, etc. The kits may also include reagents, for example, test compounds, buffers, media (e.g., cell growth media), cells, etc. Test compounds may include known compounds or newly discovered compounds, for example, combinatorial libraries of compounds. One or more of the kits of the disclosure may be packaged together, for example, a kit for assessing the efficacy of a treatment for a neurological disorder may be packaged with a kit for monitoring the progress of a subject being treated for a neurological disorder according to the disclosure.

The present methods can be performed on cells in culture, e.g. in vitro or ex vivo, or on cells present in an animal subject, e.g., in vivo. Compounds of the disclosures can be initially tested in vitro using primary cultures.

Alternatively, the effects of compound of the disclosure can be characterized in vivo using animals models. One skilled in the art, provided with the present disclosure, will readily understand that analogous screening methods for the same or a similar library of compounds could now be usefully applied to different neurological conditions in which the same or similar underlying pathologic mechanism is known or suspected to be a factor in the development or manifestation of the disease.

The recitation of a listing of chemical groups in any definition of a variable herein includes definitions of that variable as any single group or combination of listed groups. The recitation of an embodiment for a variable herein includes that embodiment as any single embodiment or in combination with any other embodiments or portions thereof. The recitation of an embodiment herein includes that embodiment as any single embodiment or in combination with any other embodiments or portions thereof.

The present disclosure provides methods of modulating (e.g., inhibiting or increasing) the activity (e.g., aberrant activity, or undesired activity, such as increased or decreased activity) of a PACAP-receptor. The present disclosure provides methods of modulating (e.g., inhibiting or increasing) the activity (e.g., aberrant activity, such as increased or decreased activity) of a PACAP-receptor in a subject. The present disclosure also provides methods for the treatment of a wide range of diseases, such as diseases associated with the aberrant activity (e.g., increased or decreased activity) of a PACAP-receptor, e.g., neurological diseases or disorders (e.g. stress-related disorders (e.g. chronic stress, post-traumatic stress disorder (PTSD), panic disorder, anxiety, or general anxiety disorder), eating disorders (e.g. orthorexia, or anorexia nervosa), pain-related disorders (e.g. chronic pain, arthritis (e.g., rheumatoid arthritis), inflammatory pain, musculoskeletal pain, primary headache disorders, or migraines), panic disorders, panic attacks, or addiction (e.g. addiction to a substance (e.g. addiction to cocaine, amphetamines, methamphetamine, methylphenidate, nicotine, alcohol, prescription medication, marijuana, tobacco, food, or an opioid selected from heroin, fentanyl, codeine, hydrocodone, morphine, oxycodone, hydromorphone, and methadone)).

### EXAMPLES

In order that the present disclosure may be more fully understood, the following examples are set forth. The synthetic and biological examples described in this application are offered to illustrate the compounds, pharmaceutical compositions, and methods provided herein and are not to be construed in any way as limiting their scope.

### Example 1. Biological Procedures

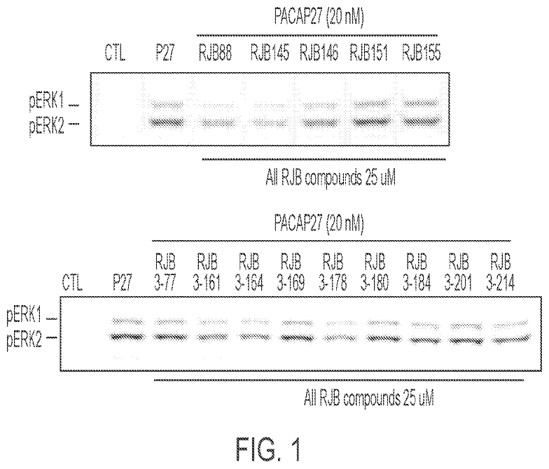

For FIG. 1, stable PAC1 receptor HEK cell lines were pretreated with 25 μM of compounds of the disclosure to evaluate inhibition of 20 nM PACAP-stimulated ERK activation by Western blot analyses.

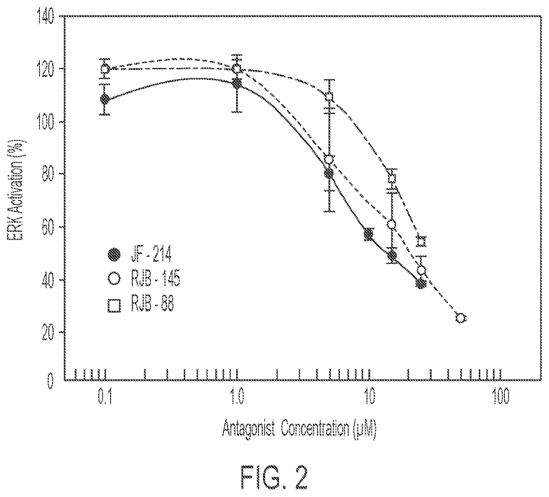

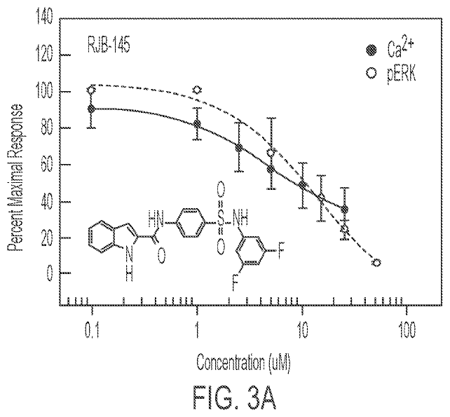

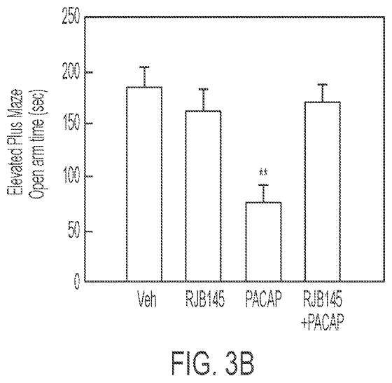

For FIG. 2, PAC1 receptor HEK cells were pretreated with different concentrations of the indicated compound to demonstrate dose-dependent inhibition of 20 nM PACAP-stimulated ERK activation. RJB-145 demonstrated an apparent IC₅₀ of approximately 10 μM which is comparable to that for a previously identified antagonist JF-214. RJB-88 appeared less potent with an IC₅₀ of approximately 25 μM.

### Example 2. Biological Results

As depicted in FIG. 1, RJB-145 and related isomers demonstrate efficacy in blocking PACAP/PAC1 receptor stimulated ERK activation.

As shown in FIG. 2, RJB-145 dose-dependent inhibition of PACAP-stimulated ERK activation. RJB-145 demonstrated an apparent IC₅₀ of approximately 10 μM which is comparable to that for a previously identified antagonist JF-214. RJB-88 appeared less potent with an IC₅₀ of approximately 25 μM.

### Example 3. Synthesis

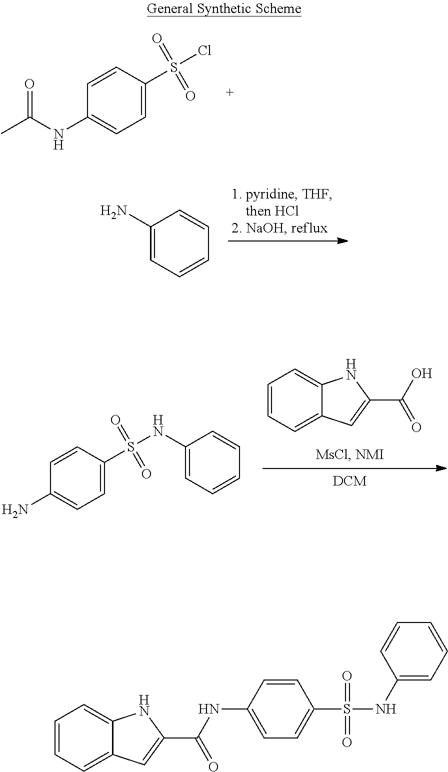

### Synthesis of Benzenesulfonyl Chloride Derivatives

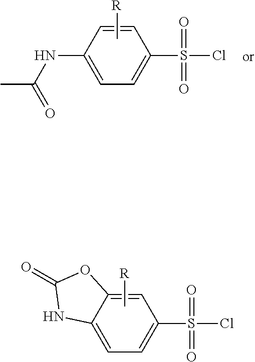

The following reaction procedure was conducted without solvent. The requisite substituted N-acetyl-aniline or benzoxazolinone (1 eq) was cooled to 0° C. and chlorosulfonic acid (7.5 eq) was slowly added dropwise at 0° C. The mixture was allowed to warm to room temperature and was stirred for 2-3 hours, at which point it was added dropwise to ice water to give the product as a precipitate which was collected via vacuum filtration.

### Synthesis of RJB-2-142

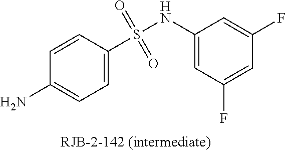 [SMILES: Nc1ccc(S(=O)(=O)Nc2cc(F)cc(F)c2)cc1]

The RJB-2-142 acetyl-protected intermediate was dissolved in 20% NaOH solution (4 ml/mmol) and was heated at reflux for 2 hours. The reaction mixture was cooled to room temperature and brought to pH=7 by the addition of 2M HCl, which resulted in the formation of a precipitate. The mixture was cooled to 0° C. and the solid product was collected via vacuum filtration.

### Synthesis of RJB-2-142 (acetyl-protected intermediate)

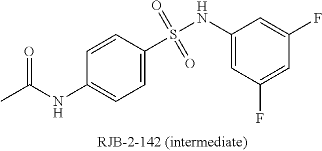 [SMILES: CC(=O)Nc1ccc(S(=O)(=O)Nc2cc(F)cc(F)c2)cc1]

Pyridine (1.2 eq) was added to a solution 3,5-difluoroaniline (1.2 eq) in dry tetrahydrofuran (0.5M) at room temperature and the mixture was allowed to stir for 5 hours. N-Acetylsulfanilyl chloride (1 eq) was added and the reaction was kept at room temperature for 18 hours. The solvent was evaporated and 0.5M HCl (2 ml/mmol) was added and the solid N-acetyl protected intermediate was then collected via vacuum filtration.

### Synthesis of RJB-2-145

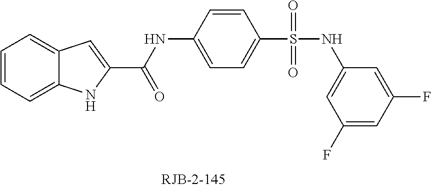 [SMILES: O=C(Nc1ccc(S(=O)(=O)Nc2cc(F)cc(F)c2)cc1)c1cc2ccccc2[nH]1]

1-Methylimidazole (3 eq) and methane sulfonyl chloride (1.5 eq) were added sequentially to a 0° C. solution of indole-2-carboxylic acid (1 eq) in dry dichloromethane (0.03M). The reaction was removed from the cooling bath and stirred for 15 minutes at RT. The reaction was cooled to 0° C. and RJB-2-142 (1 eq) was added. The mixture was then heated at reflux for 6.5 hours, cooled to RT and water was added which caused a precipitate to form. The product was collected by vacuum filtration.

### Results

Among new candidate scaffolds provided herein, one new small molecule RJB-145 shows efficacy and potency in blocking PACAP/PAC1 receptor-stimulated ERK activation in our stable human HEK PAC1 receptor cell line (FIGS. 1 and 2). RJB-145 contains a sulfanilamide core flanked by an indole and it maintains the 3,5-difluorobenzyl group that showed particular efficacy in JF-214. Again, the presence of fluorine or other electron withdrawing groups at the 3 and 5 positions of the aromatic ring appear to be relevant for potency. These heteroatoms could take advantage of a hydrogen bonding interactions within the binding pocket, or the electron deficient aryl ring may be involved in π-stacking or cation-π interactions within the receptor. Without the fluorine groups, RJB-88 appeared less potent (FIGS. 1 and 2). Substituted aryl, substituted heteroaryl including but not limited to indole derivatives, or substituted alkyl rings in place of the indole moiety may also increase potency through additional interactions with the receptor.

Compared to JF-214, RJB-145 demonstrates near equipotency in blocking PACAP/PAC1 receptor-stimulated ERK activation. Using 20 nM PACAP for near maximal ERK activation, RJB-145 demonstrated an apparent IC₅₀ of 10 μM, compared to JF-214 with an apparent IC₅₀ of 5-8 μM (FIG. 2). As with our current understanding of JF-214 signaling profile, RJB-145 also appears to be a biased antagonist in inhibiting PAC1 receptor-mediated ERK activation without significant effects on PAC1 receptor-stimulated adenylyl cyclase cAMP activation. Hence isoforms based on the RJB-145 scaffold have potential in being developed into potent and efficacious PAC1 receptor antagonists for therapeutics to ameliorate stress related behavioral psychopathologies and chronic pain disorders.

### REFERENCES

Akerman S, Goadsby P J. Neuronal PAC1 receptors mediate delayed activation and sensitization of trigeminocervical neurons: Relevance to migraine. Sci Transl Med. 2015, 7:308ra157.
Almli L M, Mercer K B, Kerley K, Feng H, Bradley B, Conneely K N, Ressler K J.. ADCYAP1R1 genotype associates with post-traumatic stress symptoms in highly traumatized African-American females. Am J Med Genet B Neuropsychiatr Genet. 2013, 162B:262-72.
Arimura, A.; Somogyvári-Vigh, A.; Miyata, A.; Mizuno, K.; Coy, D. H.; Kitada, C., Tissue distribution of PACAP as determined by RIA: highly abundant in the rat brain and testes. Endocrinology 1991, 129, 2787-2789.
Asmundson G J, Katz J. Understanding the co-occurrence of anxiety disorders and chronic pain: state-of-the-art. Depress Anxiety. 2009, 26, 888-901.
Blechman, J.; Levkowitz, G., Alternative splicing of the pituitary adenylate cyclase-activating polypeptide receptor PAC1: mechanisms of fine tuning of brain activity. Front Endocrinol (Lausanne) 2013, 4, 55.
Carrasquillo Y, Gereau R W 4th. Activation of the extracellular signal-regulated kinase in the amygdala modulates pain perception. J Neurosci. 2007, 2, 1543-51.
Chen W, Boutaoui N, Brehm J M, Han Y Y, Schmitz C, Cressley A, et al. (2013): ADCYAP1R1 and asthma in Puerto Rican children. Am J Respir Crit Care Med, 187, 584-588.
Edvinsson L, Tajti J, Szalardy L, Vécsei L. PACAP and its role in primary headaches. J Headache Pain. 2018, 19:21.
Hammack, S. E.; Cheung, J.; Rhodes, K. M.; Schutz, K. C.; Falls, W. A.; Braas, K. M.; May, V., Chronic stress increases pituitary adenylate cyclase-activating peptide (PACAP) and brain-derived neurotrophic factor (BDNF) mRNA expression in the bed nucleus of the stria terminalis (BNST): roles for PACAP in anxiety-like behavior. Psychoneuroendocrinology 2009, 34, 833-843.
Hammack, S. E.; May, V., Pituitary adenylate cyclase activating polypeptide in stress-related disorders: data convergence from animal and human studies. Biol Psychiatry 2015, 78 (3), 167-77.
Harmar, A. J.; Fahrenkrug, J.; Gozes, I.; Laburthe, M.; May, V.; Pisegna, J. R.; Vaudry, D.; Vaudry, H.; Waschek, J. A.; Said, S. I., Pharmacology and functions of receptors for vasoactive intestinal peptide and pituitary adenylate cyclase-activating polypeptide: IUPHAR review 1. Br J Pharmacol. 2012, 166, 4-17.
Johnson, G. C.; Parsons, R. L.; May, V.; Hammack, S. E., Pituitary adenylate cyclase-activating polypeptide-induced PAC1 receptor internalization and recruitment of MEK/ERK signaling enhance excitability of dentate gyrus granule cells. Am J Physiol Cell Physiol. 2020a, 318, C870-C878.
Johnson, G. C.; Parsons, R.; May, V.; Hammack, S. E., The Role of Pituitary Adenylate Cyclase-Activating Polypeptide (PACAP) Signaling in the Hippocampal Dentate Gyrus. Front Cell Neurosci 2020b, 14, 111.
Jovanovic T, Norrholm S D, Davis J, Mercer K B, Almli L, Nelson A, Cross D, Smith A, Ressler K J, Bradley B. PAC1 receptor (ADCYAP1R1) genotype is associated with dark-enhanced startle in children. Mol Psychiatry 2013, 18:742-3.
Karsan N, Goadsby P J. CGRP mechanism antagonists and migraine management. Curr Neurol Neurosci Rep. 2015, 15:25.
Lezak K R, Roman C W, Braas K M, Schutz K C, Falls W A, Schulkin J, May V, Hammack S E., Regulation of bed nucleus of the stria terminalis PACAP expression by stress and corticosterone. J Mol Neurosci. 2014, 54, 477-84.
May V, Johnson G C, Hammack S E, Braas K M, Parsons R L. PAC1 receptor internalization and endosomal MEK/ERK activationils essential for PACAP-mediated neuronal excitability. J Mol Neurosci. 71, 1536-1542.
May, V.; Buttolph, T. R.; Girard, B. M.; Clason, T. A.; Parsons, R. L., PACAP-induced ERK activation in HEK cells expressing PAC1 receptors involves both receptor internalization and PKC signaling. Am J Physiol Cell Physiol. 2014, 306, C1068-C1079.
May, V.; Parsons, R. L., G protein-coupled receptor endosomal signaling and regulation of neuronal excitability and stress responses: signaling options and lessons from the PAC1 receptor. J Cell Physiol. 2017, 232, 698-706.
McFarlane A C, Atchison M, Rafalowicz E, Papay P. Physical symptoms in post-traumatic stress disorder. J Psychosom Res. 1994, 38, 715-26.
McWilliams A L, Cox B J, Enns M W, Mood and anxiety disorders associated with chronic pain: an examination in a nationally representative sample. Pain. 2003, 106:127-33.
Miles, O. W.; Thrailkill, E. A.; Linden, A. K.; May, V.; Bouton, M. E.; Hammack, S. E., Pituitary adenylate cyclase-activating peptide in the bed nucleus of the stria terminalis mediates stress-induced reinstatement of cocaine seeking in rats. Neuropsychopharmacology 2018, 43 (5), 978-986.
Missig, G.; Mei, L.; Vizzard, M. A.; Braas, K. M.; Waschek, J. A.; Ressler, K. J.; Hammack, S. E.; May, V., Parabrachial Pituitary Adenylate Cyclase-Activating Polypeptide Activation of Amygdala Endosomal Extracellular Signal-Regulated Kinase Signaling Regulates the Emotional Component of Pain. Biol Psychiatry 2017, 81 (8), 671-682.
Missig, G.; Roman, C. W.; Vizzard, M. A.; Braas, K. M.; Hammack, S. E.; May, V., Parabrachial nucleus (PBn) pituitary adenylate cyclase activating polypeptide (PACAP) signaling in the amygdala: implication for the sensory and behavioral effects of pain. Neuropharmacology 2014, 86, 38-48.
Miyata, A.; Arimura, A.; Dahl, R. R.; Minamino, N.; Uehara, A.; Jiang, L.; Culler, M. D.; Coy, D. H., Isolation of a novel 38 residue-hypothalamic polypeptide which stimulates adenylate cyclase in pituitary cells. Biochem Biophys Res Commun. 1989, 164, 567-574.
Miyata, A.; Jiang, L.; Dahl, R. R.; Kikada, C.; Kubo, K.; Fujino, M.; Minamino, N.; Arimura, A., Isolation of a neuropeptide corresponding to the N-terminal 27 residues of the pituitary adenylate cyclase activating polypeptide with 38 residues (PACAP38). Biochem Biophys Res Commun 1990, 170, 643-648.
Moeller-Bertram, T.; Keltner, J.; Strigo, I. A., Pain and post traumatic stress disorder—review of clinical and experimental evidence. Neuropharmacology 2012, 62 (2), 586-97.
Norman, S. B.; Stein, M. B.; Dimsdale, J. E.; Hoyt, D. B., Pain in the aftermath of trauma is a risk factor for post-traumatic stress disorder. Psychol Med 2008, 38 (4), 533-42.
Otis J D, Keane T M, Kerns R D. An examination of the relationship between chronic pain and post-traumatic stress disorder. J Rehabil Res Dev. 2003, 40, 397-405.
Pohlack, S. T.; Nees, F.; Ruttorf, M.; Cacciaglia, R.; Winkelmann, T.; Schad, L. R.; Witt, S. H.; Rietschel, M.; Flor, H., Neural Mechanism of a Sex-Specific Risk Variant for Posttraumatic Stress Disorder in the Type I Receptor of the Pituitary Adenylate Cyclase Activating Polypeptide. Biol Psychiatry 2015, 78 (12), 840-7.
Ressler, K. J.; Mercer, K. B.; Bradley, B.; Jovanovic, T.; Mahan, A.; Kerley, K.; Norrholm, S. D.; Kilaru, V.; Smith, A. K.; Myers, A. J.; Ramirez, M.; Engel, A.; Hammack, S. E.; Toufexis, D.; Braas, K. M.; Binder, E. B.; May, V., Post-traumatic stress disorder is associated with PACAP and the PAC1 receptor. Nature 2011, 470 (7335), 492-7.
Roman, C. W.; Lezak, K. R.; Hartsock, M. J.; Falls, W. A.; Braas, K. M.; Howard, A. B.; Hammack, S. E.; May, V., PAC1 receptor antagonism in the bed nucleus of the stria terminalis (BNST) attenuates the endocrine and behavioral consequences of chronic stress. Psychoneuroendocrinology 2014, 47, 151-65.
Scioli-Salter E R, Forman D E, Otis J D, Gregor K, Valovski I, Rasmusson A M. The shared neuroanatomy and neurobiology of comorbid chronic pain and PTSD: therapeutic implications. Clin J Pain. 2015, 31, 363-74.
Sherwood, N. M.; Krueckl, S. L.; McRory, J. E., The origin and function of the pituitary adenylate cyclase-activating polypeptide (PACAP)/glucagon superfamily. Endocr Rev. 2000, 21, 619-70.
Spengler D, Waeber C, Pantaloni C, Holsboer F, Bockaert J, Seeburg P H, Journot L., Differential signal transduction by five splice variants of the PACAP receptor. Nature. 1993, 365, 170-5.
Stroth, N.; Eiden, L. E., Stress hormone synthesis in mouse hypothalamus and adrenal gland triggered by restraint is dependent on pituitary adenylate cyclase-activating polypeptide signaling. Neuroscience 2010, 165 (4), 1025-30.
Syed A U, Koide M, Braas K M, May V, Wellman G C. Pituitary adenylate cyclase-activating polypeptide (PACAP) potently dilates middle meningeal arteries: implications for migraine. J Mol Neurosci. 2012, 48, 574-83.
Tepper S J. History and Review of anti-Calcitonin Gene-Related Peptide (CGRP) Therapies: From Translational Research to Treatment. Headache. 2018, 58 Suppl 3:238-275.
Uddin M, Chang S C, Zhang C, Ressler K, Mercer K B, Galea S, Keyes K M, McLaughlin K A, Wildman D E, Aiello A E, Koenen K C. Adcyaplrl genotype, posttraumatic stress disorder, and depression among women exposed to childhood maltreatment. Depress Anxiety, 2013, 30:251-8.
Vaudry, D.; Falluel-Morel, A.; Bourgault, S.; Basille, M.; Burel, D.; Wurtz, O.; Fournier, A.; Chow, B. K.; Hashimoto, H.; Galas, L.; Vaudry, H., Pituitary adenylate cyclase-activating polypeptide and its receptors: 20 years after the discovery. Pharmacol Rev. 2009, 61, 283-357.
Wang L, Cao C, Wang R, Qing Y, Zhang J, Zhang X Y (2013): PAC1 receptor (ADCYAP1R1) genotype is associated with PTSD's emotional numbing symptoms in Chinese earthquake survivors. Journal of affective disorders. 150, 156-159.

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or all of the group members are present in, employed in, or otherwise relevant to a given product or process.

Furthermore, the disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the disclosure, or aspects described herein, is/are referred to as comprising particular elements and/or features, certain embodiments described herein or aspects described herein consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included.

Furthermore, unless otherwise indicated or otherwise evident from the context and understanding of one of ordinary skill in the art, values that are expressed as ranges can assume any specific value or sub-range within the stated ranges in different embodiments described herein, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present disclosure that falls within the prior art may be explicitly excluded from any one or more of the claims. Because such embodiments are deemed to be known to one of ordinary skill in the art, they may be excluded even if the exclusion is not set forth explicitly herein. Any particular embodiment described herein can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present disclosure, as defined in the following claims.

What is claimed is:

1. A compound of Formula (0): 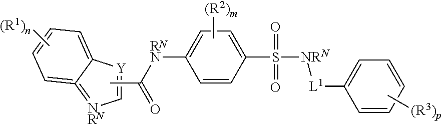
or a pharmaceutically acceptable salt thereof, wherein:
each R¹ is independently substituted or unsubstituted C₁₋₆ alkyl, halogen, hydroxy, substituted or unsubstituted C₁₋₆ alkoxy, —NR^{a}R^{a}, —C(═O)R^{a}, —C(═O)OR^{a}, —NR^{a}C(═O)R^{a}, —C(═O)N(R^{a})₂, —CN, or —NO₂;
each R² is independently substituted or unsubstituted C₁₋₆ alkyl, halogen, hydroxy, substituted or unsubstituted C₁₋₆ alkoxy, —NR^{a}R^{a}, —C(═O)R^{a}, —C(═O)OR^{a}, —NR^{a}C(═O)R^{a}, —C(═O)N(R^{a})₂, —CN, or —NO₂;
each R³ is independently halogen, hydroxy, —NR^{a}R^{a}, —C(═O)R^{a}, —C(═O)OR^{a}, —CN, or —NO₂;
each R^{N} is independently hydrogen or substituted or unsubstituted C₁₋₆ alkyl;
each R^{a} is independently H or substituted or unsubstituted C₁₋₆ alkyl;
Y is —CH—, —N—, or 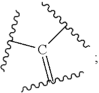 [SMILES: *C(*)=*]
L¹ is a bond or substituted or unsubstituted 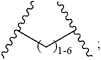
n is 0, 1, 2, 3, or 4;
m is 0, 1, 2, 3, or 4; and
p is 0, 1, 2, 3, 4, or 5.

2. The compound of claim 1, wherein the compound of Formula (0) is of Formula (0-A): 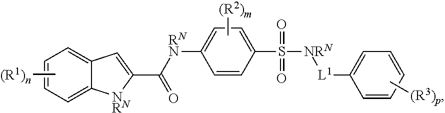
or a pharmaceutically acceptable salt thereof.

3. The compound of claim 1, wherein the compound of Formula (0) is of Formula (0-B): 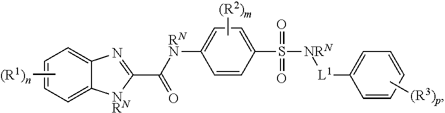
or a pharmaceutically acceptable salt thereof.

4. The compound of claim 1, wherein the compound of Formula (0) is of Formula (0-C): 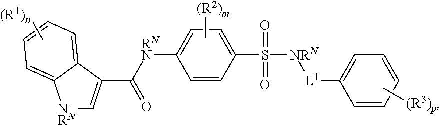
or a pharmaceutically acceptable salt thereof.

5. The compound of claim 1, wherein the compound of Formula (0) is of Formula (0-D): 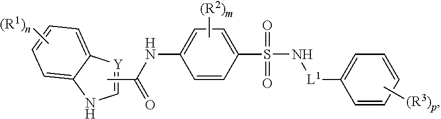
or a pharmaceutically acceptable salt thereof.

6. The compound of claim 1, wherein the compound of Formula (0) is of Formula (0-E): 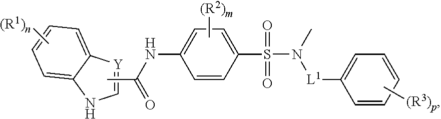
or a pharmaceutically acceptable salt thereof.

7. The compound of any one of claims 1-6, wherein L¹ is a bond.

8. The compound of any one of claims 1-6, wherein L¹ is

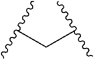 [SMILES: *C*]

9. The compound of claim 1, wherein the compound is a compound of Formula (I): 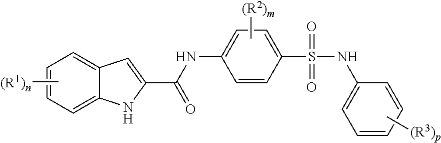
or a pharmaceutically acceptable salt thereof, wherein:
each R¹ is independently substituted or unsubstituted C₁₋₆ alkyl, halogen, hydroxy, substituted or unsubstituted C₁₋₆ alkoxy, —NR^{a}R^{a}, —C(═O)R^{a}, —C(═O)OR^{a}, —NR^{a}C(═O)R^{a}, —C(═O)N(R^{a})₂, —CN, or —NO₂;
each R² is independently substituted or unsubstituted C₁₋₆ alkyl, halogen, hydroxy, substituted or unsubstituted C₁₋₆ alkoxy, —NR^{a}R^{a}, —C(═O)R^{a}, —C(═O)OR^{a}, —NR^{a}C(═O)R^{a}, —C(═O)N(R^{a})₂, —CN, or —NO₂;
each R³ is independently halogen, hydroxy, —NR^{a}R^{a}, —C(═O)R^{a}, —C(═O)OR^{a}, —CN, or —NO₂;
each R^{a} is independently H or substituted or unsubstituted C₁₋₆ alkyl;
n is 0, 1, 2, 3, or 4;
m is 0, 1, 2, 3, or 4; and
p is 0, 1, 2, 3, 4, or 5.

10. The compound of claim 1 or 9, wherein:
each R² is independently substituted or unsubstituted C₁₋₆ alkyl, halogen, hydroxy, substituted or unsubstituted C₁₋₆ alkoxy, —C(═O)R^{a}, —C(═O)OR^{a}, —NR^{a}C(═O)R^{a}, —C(═O)N(R^{a})₂, —CN, or —NO₂;
each R³ is independently halogen, hydroxy, —C(═O)R^{a}, —C(═O)OR^{a}, —CN, or —NO₂;
each R^{a} is independently H or substituted or unsubstituted C₁₋₆ alkyl;
n is 0;
m is 0, 1, 2, 3, or 4; and
p is 0, 1, 2, 3, or 4.

11. The compound of any one of claims 1 and 9-10, wherein:
each R² is independently substituted or unsubstituted C₁₋₆ alkyl, halogen, hydroxy, or substituted or unsubstituted C₁₋₆ alkoxy;
each R³ is independently halogen, —C(═O)R^{a}, —C(═O)OR^{a}, —CN, or —NO₂;
each R^{a} is independently H or substituted or unsubstituted C₁₋₆ alkyl;
n is 0;
m is 0, 1, 2, 3, or 4; and
p is 0, 1, 2, 3, or 4.

12. The compound of any one of claims 1 and 9-11, wherein p is 1 or 2.

13. The compound of any one of claims 1 and 9-12, wherein the ring bearing R³ is of the formula:

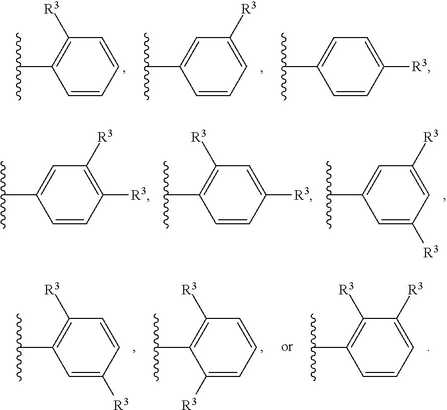

14. The compound of any one of claims 1 and 9-13, wherein the ring bearing R³ is of the formula:

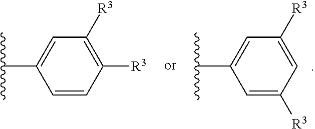

15. The compound of any one of claims 1 and 9-14, wherein the ring bearing R³ is of the formula:

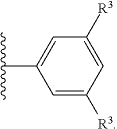

16. The compound of any one of claims 1 and 9-15, wherein the ring bearing R³ is of the formula:

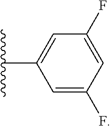 [SMILES: *c1cc(F)cc(F)c1]

17. The compound of any one of claims 1 and 9-16, wherein m is 0, 1, or 2.

18. The compound of any one of claims 1 and 9-17, wherein m is 1 or 2.

19. The compound of any one of claims 1 and 9-18, wherein each R² is independently substituted or unsubstituted C₁₋₆ alkyl, halogen, hydroxy, or substituted or unsubstituted C₁₋₆ alkoxy.

20. The compound of any one of claims 1 and 9-19, wherein each R² is independently methyl, ethyl, propyl, isopropyl, butyl, tert-butyl, halogen, hydroxy, methoxy, ethoxy, or propoxy.

21. The compound of any one of claims 1 and 9-20, wherein each R^{a} is independently unsubstituted alkyl.

22. The compound of any one of claims 1 and 9-21, wherein each R^{a} is independently unsubstituted methyl.

23. The compound of any one of claims 1 and 9-22, wherein each R^{a} is H.

24. The compound of any one of claims 1 and 9-13, wherein n is 0.

25. The compound of any one of claims 1 and 9-24, wherein the compound is of the formula: 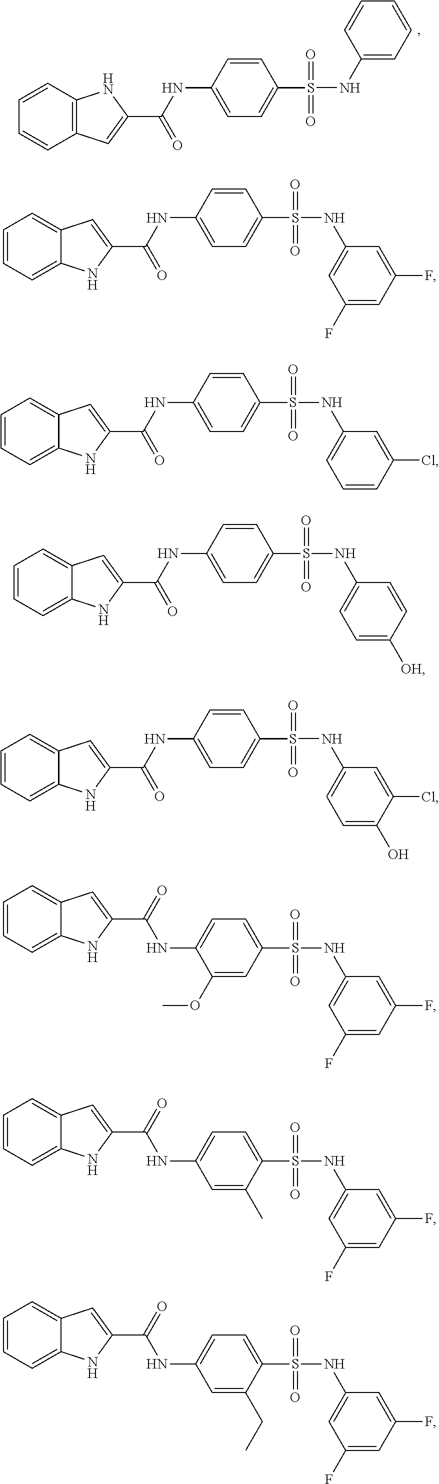 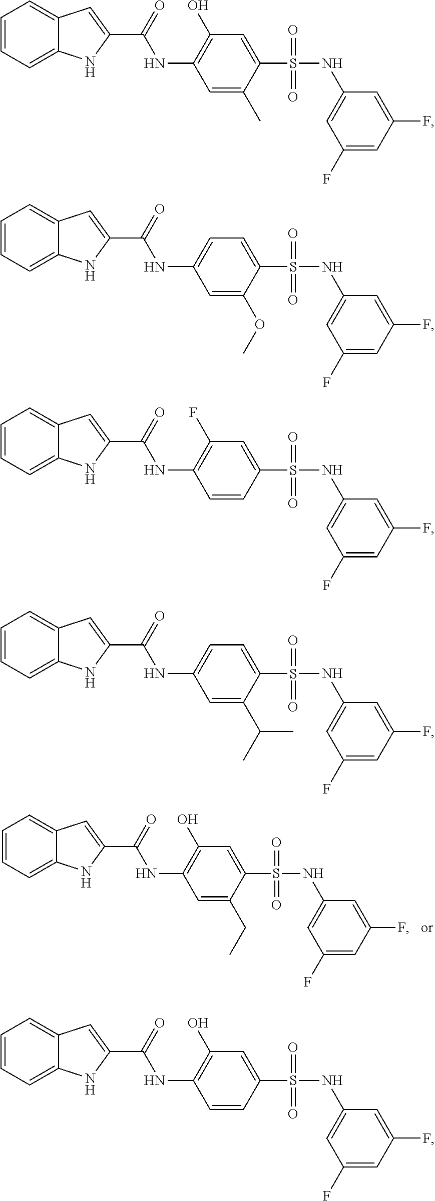 [SMILES: Cc1cc(NC(=O)c2cc3ccccc3[nH]2)c(O)cc1S(=O)(=O)Nc1cc(F)cc(F)c1 ; COc1cc(NC(=O)c2cc3ccccc3[nH]2)ccc1S(=O)(=O)Nc1cc(F)cc(F)c1 ; O=C(Nc1ccc(S(=O)(=O)Nc2cc(F)cc(F)c2)cc1F)c1cc2ccccc2[nH]1 ; CC(C)c1cc(NC(=O)c2cc3ccccc3[nH]2)ccc1S(=O)(=O)Nc1cc(F)cc(F)c1 ; CCc1cc(NC(=O)c2cc3ccccc3[nH]2)c(O)cc1S(=O)(=O)Nc1cc(F)cc(F)c1 ; O=C(Nc1ccc(S(=O)(=O)Nc2cc(F)cc(F)c2)cc1O)c1cc2ccccc2[nH]1]
or a salt thereof.

26. The compound of claim 1 or 9-25, wherein the compound is of the formula: 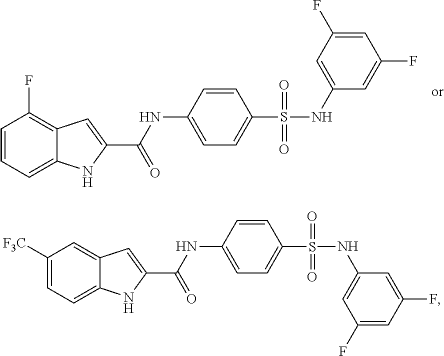 [SMILES: O=C(Nc1ccc(S(=O)(=O)Nc2cc(F)cc(F)c2)cc1)c1cc2c(F)cccc2[nH]1 ; O=C(Nc1ccc(S(=O)(=O)Nc2cc(F)cc(F)c2)cc1)c1cc2cc(C(F)(F)F)ccc2[nH]1]
or a salt thereof.

27. The compound of claim 1, wherein the compound is of the formula: 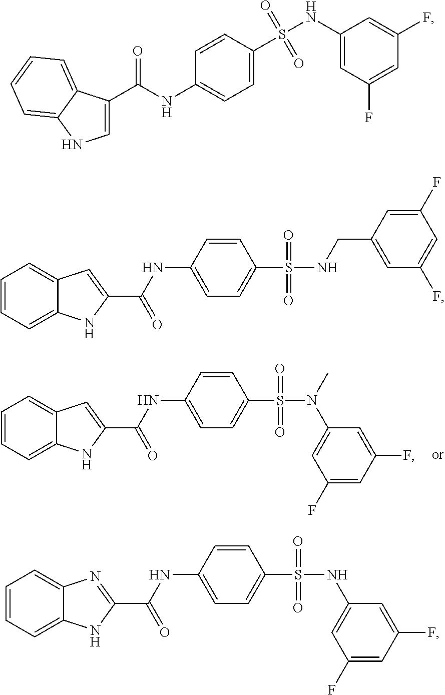
or a salt thereof.

28. A pharmaceutical composition comprising a compound of any one of claims 1-27, or a pharmaceutically acceptable salt thereof, and optionally a pharmaceutically acceptable excipient.

29. The pharmaceutical composition of claim 28 further comprising an additional pharmaceutical agent.

30. A method of treating a subject suffering from or susceptible to a neurological disease, the method comprising administering to the subject a therapeutically effective amount of a compound of any one of claims 1-27, or pharmaceutically acceptable salt thereof, or a pharmaceutical composition of claim 28 or 29.

31. The method of claim 30, wherein the neurological disease is a pain-related disorder.

32. The method of claim 30, wherein the neurological disorder is a stress-related disorder.

33. The method of claim 30, wherein the neurological disorder is addiction.

34. The method of claim 30, wherein the neurological disorder is a panic disorder.

35. The method of claim 30, wherein the neurological disorder is panic attacks.

36. The method of claim 30, wherein the neurological disorder is an eating disorder.

37. A method of treating a subject suffering from or susceptible to a pain-related disease, the method comprising administering to the subject a therapeutically effective amount of a compound of any one of claims 1-27, or pharmaceutically acceptable salt thereof, or a pharmaceutical composition of claim 28 or 29.

38. The method of claim 37, wherein the pain-related disease is chronic pain.

39. The method of claim 37, wherein the pain-related disease is a primary headache disorder.

40. The method of claim 37, wherein the primary headache disorder is a migraine.

41. The method of claim 37, wherein the pain-related disease is inflammatory pain.

42. The method of claim 37, wherein the pain-related disease is musculoskeletal pain.

43. The method of claim 37, wherein the pain-related disease is arthritis.

44. The method of claim 43, wherein the arthritis is rheumatoid arthritis.

45. A method of treating a subject suffering from or susceptible to a stress-related disease or disorder, the method comprising administering to the subject a therapeutically effective amount of a compound of any one of claims 1-27, or pharmaceutically acceptable salt thereof, or a pharmaceutical composition of claim 28 or 29.

46. The method of claim 45, wherein the stress-related disorder is chronic stress.

47. The method of claim 45, wherein the stress-related disorder is posttraumatic stress disorder (PTSD).

48. The method of claim 45, wherein the stress-related disorder is an anxiety disorder.

49. The method of claim 48, wherein the anxiety disorder is anxiety.

50. The method of claim 48, wherein the anxiety disorder is generalized anxiety disorder.

51. The method of claim 45, wherein the stress-related disorder is panic disorder.

52. A method of treating a subject suffering from or susceptible to addiction, the method comprising administering to the subject a therapeutically effective amount of a compound of any one of claims 1-27, or pharmaceutically acceptable salt thereof, or a pharmaceutical composition of claim 28 or 29.

53. The method of claim 52, wherein the addiction is addiction to a substance.

54. The method of claim 53, wherein the substance is a drug.

55. The method of claim 53, wherein the substance is food.

56. The method of claim 53, wherein the substance is cocaine, amphetamines, methamphetamine, methylphenidate, nicotine, alcohol, prescription medication, marijuana, tobacco, or an opioid selected from heroin, fentanyl, codeine, hydrocodone, morphine, oxycodone, hydromorphone, or methadone.

57. The method of claim 53, wherein the substance is cocaine.

58. The method of claim 53, wherein the substance is an opioid.

59. The method of claim 53, wherein the substance is an amphetamine.

60. The method of claim 53, wherein the substance is nicotine.

61. The method of claim 53, wherein the substance is alcohol.

62. The method of any one of claims 52-61, wherein the treatment prevents relapse of the substance abuse.

63. The method of any one of claims 52-61, wherein the treatment lowers the incidence of relapse of the substance abuse.

64. The method of claim 62 or 63, wherein the relapse is stress-induced relapse.

65. A method of treating a subject suffering from or susceptible to an eating disorder, the method comprising administering to the subject a therapeutically effective amount of a compound of any one of claims 1-27, or pharmaceutically acceptable salt thereof, or a pharmaceutical composition of claim 28 or 29.

66. The method of claim 65, wherein the eating disorder is orthorexia.

67. The method of claim 65, wherein the eating disorder is anorexia.

68. A method of treating a subject identified as in need of treatment for a pain-related disease or disorder, the method comprising administering to the subject a therapeutically effective amount of a compound of any one of claims 1-27, or pharmaceutically acceptable salt thereof, or a pharmaceutical composition of claim 28 or 29.

69. A method of treating a subject identified as in need of treatment for a stress-related disorder, the method comprising administering to the subject a therapeutically effective amount of a compound of any one of claims 1-27, or pharmaceutically acceptable salt thereof, or a pharmaceutical composition of claim 28 or 29.

70. A method of treating a subject identified as in need of treatment for a panic disorder, the method comprising administering to the subject a therapeutically effective amount of a compound of any one of claims 1-27, or pharmaceutically acceptable salt thereof, or a pharmaceutical composition of claim 28 or 29.

71. A method of treating a subject identified as in need of treatment for addiction, the method comprising administering to the subject a therapeutically effective amount of a compound of any one of claims 1-27, or pharmaceutically acceptable salt thereof, or a pharmaceutical composition of claim 28 or 29.

72. A method of treating a subject identified as in need of treatment for an eating disorder, the method comprising administering to the subject a therapeutically effective amount of a compound of any one of claims 1-27, or pharmaceutically acceptable salt thereof, or a pharmaceutical composition of claim 28 or 29.

73. The method of any one of claims 30-72 further comprising administering an additional pharmaceutical agent to the subject.

74. A kit comprising:
a compound of any one of claims 1-27, or pharmaceutically acceptable salt thereof, or a pharmaceutical composition of claim 28 or 29; and
instructions for administering to a subject or contacting a biological sample with the compound, or composition.

75. A method for modulating a pituitary adenylate cyclase activating polypeptide (PACAP) receptor, comprising contacting a PAC1 receptor with a compound of any one of claims 1-27, or pharmaceutically acceptable salt thereof, or a pharmaceutical composition of claim 28 or 29.

76. A method for modulating a PAC1 receptor, comprising contacting a PAC1 receptor with a compound of any one of claims 1-27, or pharmaceutically acceptable salt thereof, or a pharmaceutical composition of claim 28 or 29.

77. The method of claim 75 or 76, wherein the modulating is inhibiting.

78. A method of treating a disease or disorder in a subject by modulating a pituitary adenylate cyclase activating polypeptide (PACAP) receptor, the method comprising administering to the subject a therapeutically effective amount of a compound of any one of claims 1-27, or pharmaceutically acceptable salt thereof, or a pharmaceutical composition of claim 28 or 29.

79. A method of treating a disease or disorder in a subject by modulating a PAC1 receptor, the method comprising administering to the subject a therapeutically effective amount of a compound of any one of claims 1-27, or pharmaceutically acceptable salt thereof, or a pharmaceutical composition of claim 28 or 29.

80. The method of claim 78 or 79, wherein the modulating is inhibiting.
